(19) United States
(12) Patent Application Publication
(10) Pub. No.: US 2012/0129831 A1
(43) Pub. Date: May 24, 2012

(54) Substituted 3-(1,2,4-Oxadiazol-5-yl)-5-Phenylpiperidines

(75) Inventors: Dirk Heimbach, Dusseldorf (DE); Susanne Röhrig, Hilden (DE); Yolanda Cancho Grande, Leverkusen (DE); Dirk Schneider, Wuppertal (DE); Ulrich Rester, Wuppertal (DE); Eckhard Bender, Langenfeld (DE); Mark Meininghaus, Wuppertal (DE); Katja Zimmermann, Dusseldorf (DE); Dmitry Zubov, Remscheid (DE); Anja Buchmüller, Essen (DE); Georges Von Degenfeld, Leverkusen (DE); Christoph Gerdes, Koln (DE); Michael Gerisch, Wuppertal (DE); Mark Jean Gnoth, Mettmann (DE); Kersten Matthias Gericke, Wuppertal (DE); Mario Jeske, Solingen (DE)

(73) Assignee: BAYER PHARMA AKTIENGESELLSCHAFT, Berlin (DE)

(21) Appl. No.: 13/322,602

(22) PCT Filed: May 14, 2010
(86) PCT No.: PCT/EP10/02966

§ 371 Date: Feb. 9, 2012

(30) Foreign Application Priority Data
May 27, 2009 (DE) 102009022897.7

(51) Int. Cl. A61K 31/454; A61K 31/4545; A01N 1/02; A61P 9/00; A61P 35/00; C07D 413/14; A61P 7/02

(52) U.S. Cl. 514/210.18; 546/209; 546/187; 514/316; 435/2

(57) ABSTRACT

The invention relates to novel substituted piperidines, to processes for preparation thereof, to the use thereof for treatment and/or prophylaxis of diseases and to the use thereof for production of medicaments for treatment and/or prophylaxis of diseases, especially of cardiovascular disorders and tumour disorders.

The invention relates to novel substituted piperidines, to processes for preparation thereof, to the use thereof for treatment and/or prophylaxis of diseases and to the use thereof for production of medicaments for treatment and/or prophylaxis of diseases, especially of cardiovascular disorders and tumour disorders.

Thrombocytes (blood platelets) are a significant factor both in physiological haemostasis and in thromboembolic disorders. In the arterial system in particular, platelets are of central importance in the complex interaction between blood components and the wall of the vessel. Unwanted platelet activation may, through formation of platelet-rich thrombi, result in thromboembolic disorders and thrombotic complications with life-threatening conditions.

One of the most potent platelet activators is the blood coagulation protease thrombin, which is formed at injured blood vessel walls and which, in addition to fibrin formation, leads to the activation of platelets, endothelial cells and mesenchymal cells (Vu T K H, Hung D T, Wheaton V I, Coughlin S R, Cell 1991, 64, 1057-1068). In platelets in vitro and in animal models, thrombin inhibitors inhibit platelet aggregation and the formation of platelet-rich thrombi. In man, arterial thromboses can be prevented or treated successfully with inhibitors of platelet function and thrombin inhibitors (Bhatt D L, Topol E J, Nat. Rev. Drug Discov. 2003, 2, 15-28). Therefore, there is a high probability that antagonists of thrombin action on platelets will reduce the formation of thrombi and the occurrence of clinical sequelae such as myocardial infeaction and stroke. Other cellular effects of thrombin, for example on endothelial cells and smooth-muscle cells of vessels, on leukocytes and on fibroblasts, are possibly responsible for inflammatory and proliferative disorders.

At least some of the cellular effects of thrombin are mediated via a family of G-protein-coupled receptors (Protease Activated Receptors, PARs), the prototype of which is the PAR-1 receptor. PAR-1 is activated by bindung of thrombin and proteolytic cleavage of its extracellular N-terminus. The proteolysis exposes a new N-terminus having the amino acid sequence SFLLRN . . . , which, as an agonist (“tethered ligand”) leads to intramolecular receptor activation and transmission of intracellular signals. Peptides derived from the tethered-ligand sequence can be used as agonists of the receptor and, on platelets, lead to activation and aggregation. Other proteases are likewise capable of activating PAR-1, including, for example, plasmin, factor VIIa, factor Xa, trypsin, activated protein C (aPC), tryptase, cathepsin G, proteinase 3, granzyme A, elastase and matrix metalloprotease 1 (MMP-1).

In contrast to the inhibition of protease activity of thrombin with direct thrombin inhibitors, blockade of PAR-1 should result in an inhibition of platelet activation without reduction of the coagulability of the blood (anticoagulation).

Antibodies and other selective PAR-1 antagonists inhibit the thrombin-induced aggregation of platelets in vitro at low to medium thrombin concentrations (Kahn M L, Nakanishi-Matsui M, Shapiro M J, Ishihara H, Coughlin S R, J. Clin. Invest. 1999, 103, 879-887). A further thrombin receptor with possible significance for the pathophysiology of thrombotic processes, PAR-4, was identified on human and animal platelets. In experimental thromboses in animals having a PAR expression pattern comparable to humans, PAR-1 antagonists reduce the formation of platelet-rich thrombi (Derian C K, Damiano B P, Addo M F, Darrow A L, D'Andrea M R, Nedelman M, Zhang H-C, Maryanoff B E, Andrade-Gordon P, J. Pharmacol. Exp. Ther. 2003, 304, 855-861).

In the last few years, a large number of substances have been examined for their platelet function-inhibiting action; but only a few platelet function inhibitors have been found to be useful in practice. There is therefore a need for pharmaceuticals which specifically inhibit an increased platelet reaction without significantly increasing the risk of bleeding, and hence reduce the risk of thromboembolic complications.

Effects of thrombin which are mediated via the PAR-1 receptor affect the progression of disease during and after coronary artery bypass graft (CABG) and other operations and especially operations with extracorporeal circulation (for example heart-lung machine). During the course of the operation, there may be bleeding complications owing to pre- or intraoperative medication with coagulation-inhibiting and/or platelet-inhibiting substances. For this reason, for example, medication with clopidogrel has to be interrupted several days prior to a CABG. Moreover, as mentioned, disseminated intravascular coagulation or consumption coagulopathy (DIC) may develop (for example owing to the extended contact between blood and synthetic surfaces in the case of use of extracorporeal circulation or during blood transfusions), which in turn can lead to bleeding complications. Later, there is frequently restenosis of the venous or arterial bypasses grafted (which may even result in occlusion) owing to thrombosis, intimafibrosis, arteriosclerosis, angina pectoris, myocardial infarction, heart failure, arrhythmias, transitory ischaemic attack (TIA) and/or stroke.

In man, the PAR-1 receptor is also expressed in other cells including, for example, endothelial cells, smooth muscle cells and tumour cells. Malignant tumour disorders (cancer) have a high incidence and are generally associated with high mortality. Current therapies achieve full remission in only a fraction of patients and are typically associated with severe side effects. There is therefore a great need for more effective and safer therapies. The PAR-1 receptor contributes to cancer generation, growth, invasiveness and metastasis. Moreover, PAR-1 expressed on endothelial cells mediates signals resulting in vascular growth (“angiogenesis”), a process which is vital for allowing a tumour to grow larger than about 1 mm³. Angiogenesis also contributes to the genesis or worsening of other disorders including, for example, haematopoetic cancer disorders, macular degeneration, which leads to blindness, and diabetic retinopathy, inflammatory disorders, such as rheumatoid arthritis and colitis.

Sepsis (or septicaemia) is a frequent disorder with high mortality. Initial symptoms of sepsis are typically unspecific (for example fever, reduced general state of health); however, there may later be generalized activation of the coagulation system (“disseminated intravascular coagulation” or “consumption coagulopathy” (DIC)) with the formation of microthrombi in various organs and secondary bleeding complications. DIC may also occur independently of a sepsis, for example in the course of operations or in the event of tumour disorders.

Treatment of sepsis consists firstly in the rigorous elimination of the infectious cause, for example by operative removal of the focus and antibiosis. Secondly, it consists in temporary intensive medical support of the affected organ systems. Treatments of the different stages of this disease have been described, for example, in the following publication (Dellinger et al., Crit. Care Med. 2004, 32, 858-873). There are no proven effective treatments for DIC.

It is therefore an object of the present invention to provide novel PAR-1 antagonists for treatment of disorders, for example cardiovascular disorders and thromboembolic disorders, and also tumour disorders, in humans and animals.

WO 2006/012226, WO 2006/020598, WO 2007/038138, WO 2007/130898, WO 2007/101270 and US 2006/0004049 describe structurally similar piperidines as 11-β HSD1 inhibitors for treatment of diabetes, thromboembolic disorders and stroke, among other disorders.

The invention provides compounds of the formula

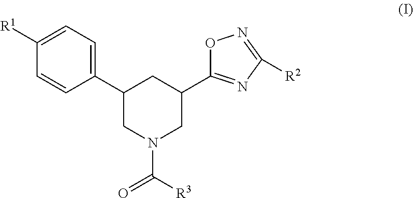

in which
R¹ is trifluoromethyl, trifluoromethoxy or ethyl,
R² is 2-methoxyeth-1-yl, 2-ethoxyeth-1-yl or cyclopropyl,
R³ is a group of the formula

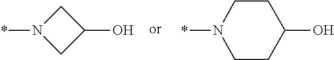

where
* is the point of attachment to the carbonyl group,
   and their salts, their solvates and the solvates of their salts.

Inventive compounds are the compounds of the formula (I) and their salts, solvates and solvates of the salts; the compounds, encompassed by formula (I), of the formulae below and their salts, solvates and solvates of the salts, and the compounds encompassed by formula (I) specified below as working examples and their salts, solvates and solvates of the salts, if the compounds, encompassed by formula (I), below are not already salts, solvates and solvates of the salts.

Depending on their structure, the inventive compounds may exist in stereoisomeric forms (enantiomers, diastereomers). The invention therefore encompasses the enantiomers or diastereomers and their respective mixtures. It is possible to isolate the stereoisomerically uniform constituents in a known manner from such mixtures of enantiomers and/or diastereomers.

If the inventive compounds can occur in tautomeric forms, the present invention encompasses all tautomeric forms.

In the context of the present invention, preferred salts are physiologically acceptable salts of the inventive compounds. However, also encompassed are salts which themselves are not suitable for pharmaceutical applications, but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of customary bases, such as, by way of example and with preference, alkali metal salts (for example sodium salts and potassium salts), alkaline earth metal salts (for example calcium salts and magnesium salts) and ammonium salts derived from ammonia or organic amines having 1 to 16 carbon atoms, such as, by way of example and with preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

In the context of the invention, solvates are those forms of the inventive compounds which, in the solid or liquid state, form a complex by coordination with solvent molecules. Hydrates are a specific form of the solvates in which the coordination is with water.

Moreover, the present invention also encompasses prodrugs of the inventive compounds. The term “prodrugs” encompasses compounds which themselves may be biologically active or inactive but which, during their residence time in the body, are converted to inventive compounds (for example metabolically or hydrolytically).

In the formula of the group which may be R³, the end point of the line with an * symbol alongside is not a carbon atom or a CH₂ group but is part of the bond to the atom to which R³ is bonded.

Preference is given to compounds of the formula (I) in which

R¹ is trifluoromethyl or ethyl,
R² is 2-methoxyeth-1-yl or cyclopropyl,
R³ is a group of the formula

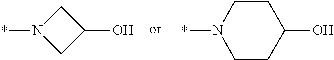

where
* is the point of attachment to the carbonyl group,
   and their salts, their solvates and the solvates of their salts.

Preference is also given to compounds of the formula (I) in which

R¹ is trifluoromethyl or ethyl,
R² is 2-methoxyeth-1-yl,
R³ is a group of the formula

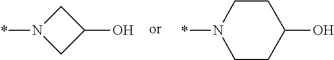

where
* is the point of attachment to the carbonyl group,
   and their salts, their solvates and the solvates of their salts.

Preference is also given to compounds of the formula (I) in which

R¹ is trifluoromethoxy or ethyl,
R² is 2-ethoxyeth-1-yl,
R³ is a group of the formula

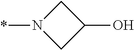

where
* is the point of attachment to the carbonyl group,
   and their salts, their solvates and the solvates of their salts.

Preference is also given to compounds of the formula (I) in which

R¹ is trifluoromethyl, trifluoromethoxy or ethyl,
R² is 2-ethoxyeth-1-yl,
R³ is a group of the formula

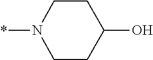

where
* is the point of attachment to the carbonyl group,
   and their salts, their solvates and the solvates of their salts.

Preference is also given to compounds of the formula (I) in which the phenyl substituent and the 1,2,4-oxadiazol-5-yl substituent bonded to the piperidine ring, are in cis positions to one another.

Preference is also given to compounds of the formula (I) in which the carbon atom to which the phenyl substituent is bonded has S configuration and the carbon atom to which the 1,2,4-oxadiazol-5-yl substituent is bonded likewise has S configuration.

Preference is also given to compounds of the formula (I) in which R¹ is trifluoromethyl.

Preference is also given to compounds of the formula (I) in which R¹ is ethyl.

Preference is also given to compounds of the formula (I) in which R² is 2-methoxyeth-1-yl.

Preference is also given to compounds of the formula (I) in which R² is 2-ethoxyeth-1-yl.

Preference is also given to compounds of the formula (I) in which

R³ is a group of the formula

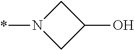

where
* is the point of attachment to the carbonyl group.

Preference is also given to compounds of the formula (I) in which

R³ is a group of the formula

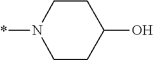

where
* is the point of attachment to the carbonyl group.

The individual radical definitions specified in the respective combinations or preferred combinations of radicals are, independently of the respective combinations of the radicals specified, also replaced as desired by radical definitions of other combinations.

Very particular preference is given to combinations of two or more of the preferred ranges mentioned above.

The invention further provides a process for preparing the compounds of the formula (I), or their salts, their solvates or the solvates of their salts, where either

[A] compounds of the formula

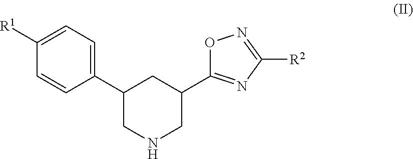

in which
R¹ and R² are each as defined above
are reacted with compounds of the formula

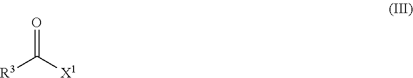

in which
R³ is as defined above and
X¹ is halogen, preferably bromine or chlorine, or hydroxyl,
or
[B] compounds of the formula (II) are reacted in the first stage with 4-nitrophenyl chloroformate and in the second stage with compounds of the formula

R³—H (IV)

in which
R³ is as defined above
or
[C] compounds of the formula

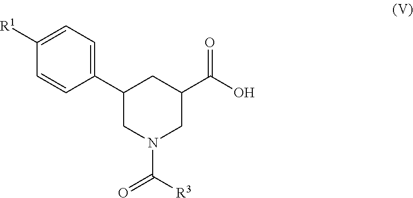

in which
R¹ and R³ are each as defined above
are reacted with compounds of the formula

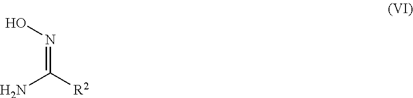

in which
R² is as defined above.

When X¹ is halogen, the reaction according to process [A] is generally effected in inert solvents, optionally in the presence of a base, preferably in a temperature range of −30° C. to 50° C. at standard pressure.

Inert solvents are, for example, tetrahydrofuran, methylene chloride, pyridine, dioxane or dimethylformamide, preference being given to methylene chloride.

Bases are, for example, triethylamine, diisopropylethylamine or N-methylmorpholine, preference being given to triethylamine or diisopropylethylamine.

When X¹ is hydroxyl, the reaction according to process [A] is generally effected in inert solvents, in the presence of a dehydrating reagent, optionally in the presence of a base, preferably in a temperature range of −30° C. to 50° C. at standard pressure.

Inert solvents are, for example, halohydrocarbons such as dichloromethane or trichloromethane, hydrocarbons such as benzene, nitromethane, dioxane, dimethylformamide or acetonitrile. It is equally possible to use mixtures of the solvents. Particular preference is given to dichloromethane or dimethylformamide.

Suitable dehydrating reagents in this context are, for example, carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethylpolystyrene (PS-carbodiimide), or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or N-hydroxysuccinimide, or mixtures of these, with bases.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine.

Preferably, the condensation is performed with HATU or with EDC in the presence of HOBt.

The compounds of the formula (III) are known or can be synthesized by known processes from the appropriate starting compounds.

The first stage reaction according to process [B] is generally effected in inert solvents, in the presence of a base, preferably in a temperature range of 0° C. to 50° C. at standard pressure.

Inert solvents are, for example, halohydrocarbons such as methylene chloride, trichloromethane, tetrachloromethane or 1,2-dichloroethane, preference being given to methylene chloride.

Bases are, for example, organic bases, such as trialkylamines, for example triethylamine, N-methylmorpholine, N-methylpiperidine, 4-dimethylaminopyridine or diisopropylethylamine, preference being given to triethylamine.

The reaction of the second stage according to process [B] is generally effected in inert solvents, in the presence of a base, optionally in a microwave, preferably in a temperature range of 50° C. to 200° C. at standard pressure to 5 bar.

Inert solvents are, for example, dimethyl sulphoxide, dimethylformamide or N-methylpyrrolidone, preference being given to dimethylformamide.

Bases are, for example, alkali metal carbonates, for example sodium carbonate or potassium carbonate, preference being given to potassium carbonate.

The compounds of the formula (IV) are known or can be synthesized by known processes from the appropriate starting compounds.

The reaction according to process [C] is generally effected in inert solvents, in the presence of a dehydrating reagent, optionally in the presence of a base, preferably in a temperature range from room temperature up to reflux of the solvents at standard pressure.

Inert solvents are, for example, halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile. It is equally possible to use mixtures of the solvents. Preference is given to dimethylformamide or a mixture of dioxane and dimethylformamide.

Suitable dehydrating reagents in this context are, for example, carbodiimides, for example N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethylpolystyrene (PS-carbodiimide), or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or benzotriazol-1-yloxytris(pyrrolidino)phosphonium hexafluorophosphate (PYBOP), or N-hydroxysuccinimide, or mixtures of these with bases.

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine, preference being given to diisopropylethylamine.

Preferably, the condensation is performed with HATU in the presence of diisopropylethylamine or alternatively only with carbonyldiimidazole.

The compounds of the formula (VI) are known or can be synthesized by known processes from the appropriate starting compounds.

The compounds of the formula (II) are known and/or can be prepared by reacting compounds of the formula

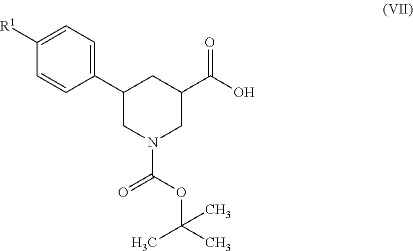

in which
R¹ is as defined above
in the first stage with compounds of the formula (VI) and in the second stage with an acid.

The first stage reaction is effected as described for process [C].

The second stage reaction is generally effected in inert solvents, preferably in a temperature range of room temperature to 60° C. at standard pressure.

Inert solvents are, for example, halohydrocarbons such as methylene chloride, trichloromethane, tetrachloromethane or 1,2-dichloroethane, or ethers such as tetrahydrofuran or dioxane, preference being given to methylene chloride.

Bases are, for example, trifluoroacetic acid or hydrogen chloride in dioxane, preference being given to trifluoroacetic acid.

The compounds of the formula (VII) are known and/or can be prepared by reacting compounds of the formula

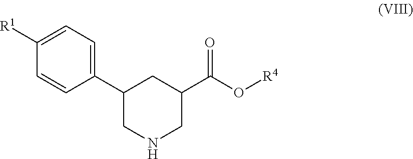

in which
R¹ is as defined above, and
R⁴ is methyl or ethyl,
in the first stage with di-tert-butyl dicarboxylate and
in the second stage with a base.

The first stage reaction is generally effected in inert solvents, in the presence of a base, preferably in a temperature range of room temperature to 50° C. at standard pressure.

Inert solvents are, for example, halohydrocarbons such as methylene chloride, trichloromethane, tetrachloromethane or 1,2-dichloroethane, preference being given to methylene chloride.

Bases are, for example, triethylamine, diisopropylethylamine or N-methylmorpholine, preference being given to triethylamine or diisopropylethylamine.

The second stage reaction is generally effected in inert solvents, in the presence of a base, preferably in a temperature range of room temperature up to reflux of the solvents at standard pressure.

Inert solvents are, for example, halohydrocarbons such as methylene chloride, trichloromethane, tetrachloromethane or 1,2-dichloroethane, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium, lithium or potassium hydroxide, or alkali metal carbonates such as caesium carbonate, sodium or potassium carbonate, preference being given to lithium hydroxide.

The compounds of the formula (VIII) are known and/or can be prepared by hydrogenating compounds of the formula

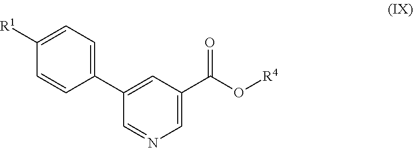

in which
R¹ and R⁴ are each as defined above.

The hydrogenation is generally effected with a reducing agent in inert solvents, optionally with addition of acid such as mineral acids and carboxylic acids, preferably acetic acid, preferably in a temperature range of room temperature up to reflux of the solvents and in a pressure range of standard pressure to 100 bar, preferably at 50-80 bar.

A preferred reducing agent is hydrogen with palladium on activated carbon, with rhodium on activated carbon, with ruthenium on activated carbon or mixed catalysts thereof, or hydrogen with palladium on alumina or with rhodium on alumina, preference being given to hydrogen with palladium on activated carbon or with rhodium on activated carbon.

Inert solvents are, for example, alcohols such as methanol, ethanol, n-propanol, isopropanol, n-butanol or tert-butanol, preference being given to methanol or ethanol.

The compounds of the formula (IX) are known and/or can be prepared by reacting compounds of the formula

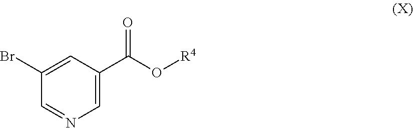

in which
R⁴ is as defined above
with compounds of the formula

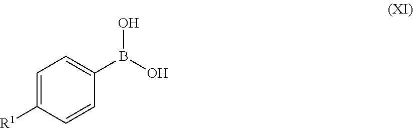

in which
R¹ is as defined above.

The reaction is generally effected in inert solvents, in the presence of a catalyst, optionally in the presence of an additional reagent, preferably in a temperature range of room temperature up to reflux of the solvent at standard pressure.

Inert solvents are, for example, ethers such as dioxane, tetrahydrofuran or 1,2-dimethoxyethane, hydrocarbons such as benzene, xylene or toluene, or other solvents such as nitrobenzene, dimethylformamide, dimethylacetamide, dimethyl sulphoxide or N-methylpyrrolidone; a little water is optionally added to these solvents. Preference is given to toluene with water or to a mixture of 1,2-dimethoxyethane, dimethylformamide and water.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate or bis(diphenylphosphinoferrocenyl)palladium(II) chloride, for example.

Additional reagents are, for example, potassium acetate, caesium, potassium or sodium carbonate, barium hydroxide, potassium tert-butoxide, caesium fluoride, potassium fluoride or potassium phosphate, preference being given to potassium fluoride or sodium carbonate.

The compounds of the formulae (X) and (XI) are known or can be synthesized by known processes from the appropriate starting compounds.

The compounds of the formula (V) are known and/or can be prepared by reacting compounds of the formula

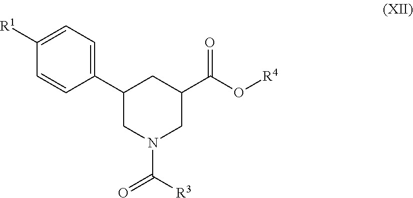

in which
R¹ and R³ are each as defined above and
R⁴ is methyl or ethyl,
with a base.

The reaction is generally effected in inert solvents, in the presence of a base, preferably in a temperature range of room temperature up to reflux of the solvents at standard pressure.

Inert solvents are, for example, halohydrocarbons such as methylene chloride, trichloromethane, tetrachloromethane or 1,2-dichloroethane, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium, lithium or potassium hydroxide, or alkali metal carbonates such as caesium carbonate, sodium or potassium carbonate, preference being given to lithium hydroxide.

The compounds of the formula (XII) are known and/or can be prepared by reacting compounds of the formula (VIII) with compounds of the formula (III).

The reaction is conducted as described for process [A].

In an alternative process, the compounds of the formula (XII) can be prepared by reacting compounds of the formula (VIII) in the first stage with 4-nitrophenyl chloroformate and in the second stage with compounds of the formula (IV).

The reaction is conducted as described for process [B].

The preparation of the compounds of the formula (I) can be illustrated by the synthesis scheme below.

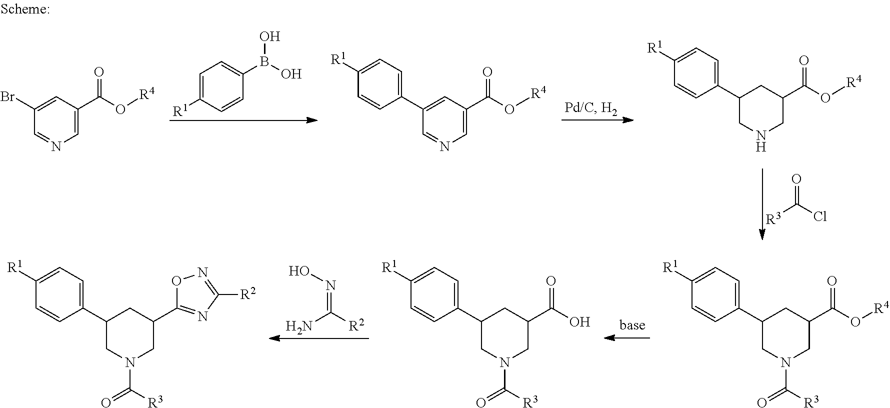

The inventive compounds exhibit an unforeseeable, useful spectrum of pharmacological and pharmacokinetic action. They are selective antagonists of the PAR-1 receptor acting in particular as platelet aggregation inhibitors, as inhibitors of endothelial proliferation and as inhibitors of tumour growth.

They are therefore suitable for use as medicaments for treatment and/or prophylaxis of diseases in man and animals.

The present invention further provides for the use of the inventive compounds for treatment and/or prophylaxis of disorders, preferably of thromboembolic disorders and/or thromboembolic complications.

“Thromboembolic disorders” in the sense of the present invention include in particular disorders such as ST-segment elevation myocardial infarction (STEMI) and non-ST-segment elevation myocardial infarction (non-STEMI), stabile angina pectoris, unstabile angina pectoris, reocclusions and restenoses after coronary interventions such as angioplasty, stent implantations or aortocoronary bypass, peripheral arterial occlusion diseases, pulmonary embolisms, deep venous thromboses and renal vein thromboses, transitory ischaemic attacks and also thrombotic and thromboembolic stroke.

The substances are therefore also suitable for prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with intravasal objects, for example artificial heart valves, catheters, intraaortic balloon counterpulsation and pacemaker probes.

Thromboembolic complications are also encountered in connection with microangiopathic haemolytic anaemias, extracorporeal circulation, for example haemodialysis, haemofiltration, ventricular assist devices and artificial hearts, and also heart valve prostheses.

Moreover, the inventive compounds are also used to influence wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, COPD, inflammatory pulmonary disorders, glomerulonephritis and inflammatory intestinal disorders, and additionally also for the prophylaxis and/or treatment of Alzheimer's disease, autoimmune disorders, Crohn's disease and ulcerative colitis.

Moreover, the inventive compounds can be used to inhibit tumour growth and metastasization, for microangiopathies, age-related macular degeneration, diabetic retinopathy, diabetic nephropathy and other microvascular disorders, and also for prevention and treatment of thromboembolic complications, for example venous thromboembolisms, for tumour patients, in particular those undergoing major surgical interventions or chemo- or radiotherapy.

The inventive compounds are additionally suitable for treatment of cancer. Cancers include: carcinomas (including breast cancer, hepatocellular carcinomas, lung cancer, colorectal cancer, cancer of the colon and melanomas), lymphomas (for example non-Hodgkin's lymphomas and mycosis fungoides), leukaemias, sarcomas, mesotheliomas, brain cancer (for example gliomas), germinomas (for example testicular cancer and ovarian cancer), choriocarcinomas, renal cancer, cancer of the pancreas, thyroid cancer, head and neck cancer, endometrial cancer, cancer of the cervix, cancer of the bladder, stomach cancer and multiple myeloma.

Moreover, PAR-1 expressed on endothelial cells mediates signals resulting in vascular growth (“angiogenesis”), a process which is vital for enabling tumour growth beyond about 1 mm³. Induction of angiogenesis is also relevant for other disorders, including disorders of the rheumatic type (for example rheumatoid arthritis), pulmonary disorders (for example pulmonary fibrosis, pulmonary hypertension, in particular pulmonary arterial hypertension, disorders characterized by pulmonary occlusion), arteriosclerosis, plaque rupture, diabetic retinopathy and wet macular degeneration.

In addition, the inventive compounds are suitable for the treatment of sepsis. Sepsis (or septicaemia) is a common disorder with high mortality. Initial symptoms of sepsis are typically unspecific (for example fever, reduced general state of health), but there may later be generalized activation of the coagulation system (“disseminated intravascular coagulation” or “consumption coagulopathy”; referred to hereinafter as “DIC”) with the formation of microthrombi in various organs and secondary bleeding complications. Moreover, there may be endothelial damage with increased permeability of the vessels and diffusion of fluid and proteins into the extravasal space. As the disorder worsens, there may be organ dysfunction or organ failure (for example kidney failure, liver failure, respiratory failure, deficits of the central nervous system and heart/circulatory failure) and even multi-organ failure. In principle, this may affect any organ; the most frequently encountered organ dysfunctions and organ failures are those of the lung, the kidney, the cardiovascular system, the coagulation system, the central nervous system, the endocrine glands and the liver. Sepsis may be associated with an “acute respiratory distress syndrome” (referred to hereinafter as ARDS). ARDS may also occur independently of sepsis. “Septic shock” is the occurrence of hypotension which has to be treated and facilitates further organ damage and is associated with a worsening of the prognosis.

Pathogens can be bacteria (gram-negative and gram-positive), fungi, viruses and/or eukaryotes. The site of entry or primary infection may be pneumonia, an infection of the urinary tract or peritonitis, for example. The infection may, but need not necessarily, be associated with bacteriaemia.

Sepsis is defined as the presence of an infection and a “systemic inflammatory response syndrome” (referred to hereinafter as “SIRS”). SIRS occurs during infections, but also during other states such as injuries, burns, shock, operations, ischaemia, pancreatitis, reanimation or tumours. The definition of ACCP/SCCM Consensus Conference Committee of 1992 (Crit. Care Med. 1992, 20, 864-874) describes the symptoms required for the diagnosis of “SIRS” and measurement parameters (including a change in body temperature, increased heart rate, breathing difficulties and changes in the blood picture). The later (2001) SCCM/ESICM/ACCP/ATS/SIS International Sepsis Definitions Conference essentially maintained the criteria, but fine-tuned details (Levy et al., Crit. Care Med. 2003, 31, 1250-1256).

DIC and SIRS may occur during sepsis, but also as a result of operations, tumour disorders, burns or other injuries. In the case of DIC, there is massive activation of the coagulation system at the surface of damaged endothelial cells, the surfaces of foreign bodies or injured extravascular tissue. As a consequence, there is coagulation in small vessels of various organs with hypoxia and subsequent organ dysfunction. A secondary effect is the consumption of coagulation factors (for example factor X, prothrombin, fibrinogen) and platelets, which reduces the coagulability of the blood and may result in heavy bleeding.

In addition, the inventive compounds can also be used for preventing coagulation ex vivo, for example for preserving blood and plasma products, for cleaning/pretreating catheters and other medical aids and instruments, including extracorporeal circulation, for coating synthetic surfaces of medical aids and instruments used in vivo or ex vivo or for platelet-containing biological samples.

The present invention further provides for the use of the inventive compounds for coating medical instruments and implants, for example catheters, prostheses, stents or artificial heart valves. The inventive compounds may be firmly attached to the surface or, for local action, be released over a certain period of time from a carrier coating into the immediate environment.

The present invention further provides for the use of the inventive compounds for treatment and/or prophylaxis of disorders, in particular of the abovementioned disorders.

The present invention further provides for the use of the inventive compounds for production of a medicament for treatment and/or prophylaxis of disorders, in particular of the abovementioned disorders.

The present invention further provides a method for treatment and/or prophylaxis of disorders, in particular of the abovementioned disorders, using a therapeutically effective amount of an inventive compound.

The present invention further provides medicaments comprising an inventive compound and one or more further active ingredients, in particular for treatment and/or prophylaxis of the abovementioned disorders. Active ingredients suitable for combinations include, by way of example and with preference:

calcium channel blockers, for example amlodipine besilate (for example Norvasc®), felodipine, diltiazem, verapamil, nifedipine, nicardipine, nisoldipine and bepridil;
iomerizine;
statins, for example atorvastatin, fluvastatin, lovastatin, pitavastatin, pravastatin, rosuvastatin and simvastatin;
cholesterol absorption inhibitors, for example ezetimibe and AZD4121;
cholesteryl ester transfer protein (“CETP”) inhibitors, for example torcetrapib;
low molecular weight heparins, for example dalteparin sodium, ardeparin, certoparin, enoxaparin, parnaparin, tinzaparin, reviparin and nadroparin;
further anticoagulants, for example warfarin, marcumar, fondaparinux;
antiarrhythmics, for example dofetilide, ibutilide, metoprolol, metoprolol tartrate, propranolol, atenolol, ajmaline, disopyramide, prajmaline, procainamide, quinidine, sparteine, aprindine, lidocaine, mexiletine, tocamide, encamide, flecamide, lorcamide, moricizine, propafenone, acebutolol, pindolol, amiodarone, bretylium tosylate, bunaftine, sotalol, adenosine, atropine and digoxin;
alpha-adrenergic agonists, for example doxazosin mesylate, terazoson and prazosin;
beta-adrenergic blockers, for example carvedilol, propranolol, timolol, nadolol, atenolol, metoprolol, bisoprolol, nebivolol, betaxolol, acebutolol and bisoprolol;
aldosterone antagonists, for example eplerenone and spironolactone;
angiotensin-converting enzyme inhibitors (“ACE inhibitors”), for example moexipril, quinapril hydrochloride, ramipril, lisinopril, benazepril hydrochloride, enalapril, captopril, spirapril, perindopril, fosinopril and trandolapril;
angiotensin II receptor blockers (“ARBs”), for example olmesartan-medoxomil, candesartan, valsartan, telmisartan, irbesartan, losartan and eprosartan;
endothelin antagonists, for example tezosentan, bosentan and sitaxsentan-sodium;
inhibitors of neutral endopeptidase, for example candoxatril and ecadotril;
phosphodiesterase inhibitors, for example milrinone, theophylline, vinpocetine, EHNA (erythro-9-(2-hydroxy-3-nonyl)adenine), sildenafil, vardenafil and tadalafil;
fibrinolytics, for example reteplase, alteplase and tenecteplase;
GP IIb/IIIa antagonists, for example integrillin, abciximab and tirofiban;
direct thrombin inhibitors, for example AZD0837, argatroban, bivalirudin and dabigatran;
indirect thrombin inhibitors, for example odiparcil;
direct and indirect factor Xa inhibitors, for example fondaparinux-sodium, apixaban, razaxaban, rivaroxaban (BAY 59-7939), KFA-1982, DX-9065a, AVE3247, otamixaban (XRP0673), AVE6324, SAR377142, idraparinux, SSR126517, DB-772d, DT-831j, YM-150, 813893, LY517717 and DU-1766;
direct and indirect factor Xa/IIa inhibitors, for example enoxaparin-sodium, AVE5026, SSR128428, SSR128429 and BIBT-986 (tanogitran);
lipoprotein-associated phospholipase A2 (“LpPLA2”) modulators;
diuretics, for example chlorthalidone, ethacrynic acid, furosemide, amiloride, chlorothiazide, hydrochlorothiazide, methylclothiazide and benzthiazide;
nitrates, for example isosorbide 5-mononitrate;
thromboxane antagonists, for example seratrodast, picotamide and ramatroban;
platelet aggregation inhibitors, for example clopidogrel, ticlopidine, cilostazol, aspirin, abciximab, limaprost, eptifibatide and CT-50547;
cyclooxygenase inhibitors, for example meloxicam, rofecoxib and celecoxib;
B-type natriuretic peptides, for example nesiritide, ularitide;
NV1FGF modulators, for example XRP0038;
HT1B/5-HT2A antagonists, for example SL65.0472;
guanylate cyclase activators, for example ataciguat (HMR1766), HMR1069, riociguat and cinaciguat;
e-NOS transcription enhancers, for example AVE9488 and AVE3085;
antiatherogenic substances, for example AGI-1067;
CPU inhibitors, for example AZD9684;
renin inhibitors, for example aliskirin and VNP489;
inhibitors of adenosine diphosphate-induced platelet aggregation, for example clopidogrel, ticlopidine, prasugrel, AZD6140, ticagrelor and elinogrel;
NHE-1 inhibitors, for example AVE4454 and AVE4890.

Antibiotic therapy: various antibiotics or antifungal medicament combinations are suitable, either as calculated therapy (before the microbial assessment has been made) or as specific therapy; fluid therapy, for example crystalloid or colloidal fluids; vasopressors, for example norepinephrine, dopamine or vasopressin; inotropic therapy, for example dobutamine; corticosteroids, for example hydrocortisone, or fludrocortisone; recombinant human activated protein C, Xigris; blood products, for example erythrocyte concentrates, platelet concentrates, erythropoietin or fresh frozen plasma; assisted ventilation in sepsis-induced acute lung injury (ALI) or acute respiratory distress syndrome (ARDS), for example permissive hypercapnia, low tidal volumes; sedation: for example diazepam, lorazepam, midazolam or propofol. Opioids: for example fentanyl, hydromorphone, morphine, meperidine or remifentanil. NSAIDs: for example ketorolac, ibuprofen or acetaminophen. Neuromuscular blockade: for example pancuronium; glucose control, for example insulin, glucose; renal replacement therapies, for example continuous veno-venous haemofiltration or intermittent haemodialysis. Low-dose dopamine for renal protection; anticoagulants, for example for thrombosis prophylaxis or for renal replacement therapies, for example unfractionated heparins, low molecular weight heparins, heparinoids, hirudin, bivalirudin or argatroban; bicarbonate therapy; stress ulcer prophylaxis, for example H2 receptor inhibitors, antacids.

Medicaments for proliferative disorders: uracil, chlormethine, cyclophosphamide, ifosfamide, melphalan, chlorambucil, pipobroman, triethylenemelamine, triethylenethiophosphoramine, busulphan, carmustine, lomustine, streptozocin, dacarbazine, methotrexate, 5-fluorouracil, floxuridine, cytarabine, 6-mercaptopurine, 6-thioguanine, fludarabine phosphate, pentostatin, vinblastine, vincristine, vindesine, bleomycin, dactinomycin, daunorubicin, doxorubicin, epirubicin, idarubicin, paclitaxel, mithramycin, deoxycoformycin, mitomycin-C, L-asparaginase, interferons, etoposide, teniposide, 17. alpha.-ethynylestradiol, diethylstilbestrol, testosterone, prednisone, fluoxymesterone, dromostanolone propionate, testolactone, megestrol acetate, tamoxifen, methylprednisolone, methyltestosterone, prednisolone, triamcinolone, chlorotrianisene, hydroxyprogesterone, aminoglutethimide, estranrustine, medroxyprogesterone acetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin (Eloxatin®), Iressa (gefmitib, Zd1839), XELODA® (capecitabine), Tarceva® (erlotinib), Azacitidine (5-azacytidine; 5-AzaC), temozolomide (Temodar®), gemcitabine (e.g. GEMZAR® (gemcitabine HCl)), vasostatin or a combination of two or more of the above.

The present invention further provides a method for prevention of blood coagulation in vitro, in particular in banked blood or biological samples containing platelets, which is characterized in that an anticoagulatory amount of the inventive compound is added.

The inventive compounds can act systemically and/or locally. For this purpose, they can be administered in a suitable way, for example, by the oral, parenteral, pulmonary, nasal, sublingual, lingual, buccal, rectal, dermal, transdermal, conjunctival, otic route or as implant or stent.

The inventive compounds can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example, tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration include preparations for injection and infusion in the form of solutions, suspensions, emulsions, lyophilizates or sterile powders.

Oral administration is preferred.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

The inventive compounds can be converted to the administration forms mentioned. This can be done in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants, for example, ascorbic acid), colours (e.g. inorganic pigments, for example, iron oxides) and masking flavours and/or odours.

The present invention further provides medicaments comprising at least one inventive compound, preferably together with one or more inert, non-toxic, pharmaceutically acceptable excipients, and their use for the purposes mentioned above.

In the case of parenteral administration, it has generally been found to be advantageous to administer amounts of about 5 to 250 mg every 24 hours to achieve effective results. In the case of oral administration the amount is about 5 to 100 mg every 24 hours.

It may nevertheless be necessary where appropriate to deviate from the stated amounts, in particular as a function of the body weight, route of administration, individual response to the active ingredient, nature of the preparation and time or interval over which administration takes place.

The percentages in the tests and examples which follow are, unless stated otherwise, percentages by weight; parts are parts by weight. Solvent ratios, dilution ratios and concentration figures for liquid/liquid solutions are based in each case on volume. “w/v” means “weight/volume”. For example, “10% w/v” means: 100 ml of solution or suspension comprise 10 g of substance.

### A) EXAMPLES

### Abbreviations

approx. approximately
CDI carbonyldiimidazole
d day(s), doublet (in NMR)
TLC thin-layer chromatography
DCI direct chemical ionization (in MS)
dd double doublet (in NMR)
DMAP 4-dimethylaminopyridine
DMF N,N-dimethylformamide
DMSO dimethyl sulphoxide
DPPA diphenyl phosphorazidate
DSC disuccinimidyl carbonate
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC-MS liquid chromatography-coupled mass spectroscopy
LDA lithium diisopropylamide
m multiplet (in NMR)
min minute(s)
MS mass spectroscopy
NMR nuclear magnetic resonance spectroscopy
PYBOP benzotriazol-1-yloxytris(pyrrolidino)phosphonium hexafluorophosphate
q quartet (in NMR)
RP reverse phase (in HPLC)
RT room temperature
Rₜ retention time (in HPLC)
s singlet (in NMR)
t triplet (in NMR)
THF tetrahydrofuran

### HPLC Methods:

Method 1A: Instrument: HP 1100 with DAD detection; column: Kromasil 100 RP-18, 60 mm×2.1 mm, 3.5 μm; eluent A: 5 ml of perchloric acid (70%)/1 of water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

### LC-MS Methods:

Method 1B: MS instrument type: Micromass ZQ; HPLC instrument type: HP 1100 Series; UV DAD; column: Phenomenex Gemini 3μ, 30 mm×3.0 mm; eluent A: 1 l of water+0.5 ml of 50% formic acid, eluent B: 1 l of acetonitrile+0.5 ml of 50% formic acid; gradient 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 2B: Instrument: Micromass QuattroPremier with Waters HPLC Acquity; column: Thermo Hypersil GOLD 1.9μ, 50 mm×1 mm; eluent A: 1 l of water+0.5 ml of 50% formic acid, eluent B: 1 l of acetonitrile+0.5 ml of 50% formic acid; gradient: 0.0 min 90% A→0.1 min 90% A→1.5 min 10% A→2.2 min 10% A; oven: 50° C.; flow rate: 0.33 ml/min; UV detection: 210 nm.

Method 3B: MS instrument type: Micromass ZQ; HPLC instrument type: Waters Alliance 2795; column: Phenomenex Synergi 2.5μ MAX-RP 100A Mercury, 20 mm×4 mm; eluent A: 1 l of water+0.5 ml of 50% formic acid, eluent B: 1 l of acetonitrile+0.5 ml of 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 4B: MS instrument type: Waters ZQ; HPLC instrument type: Waters Alliance 2795; column: Phenomenex Onyx Monolithic C18, 100 mm×3 mm; eluent A: 1 l of water+0.5 ml of 50% formic acid, eluent B: 1 l of acetonitrile+0.5 ml of 50% formic acid; gradient 0.0 min 90% A→2 min 65% A→4.5 min 5% A→6 min 5% A; flow rate: 2 ml/min; oven: 40° C.; UV detection: 210 nm.

Method 5B: Instrument: Micromass Quattro Micro MS with HPLC Agilent Series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→3.0 min 10% A→4.0 min 10% A→4.01 min 100% A→5.00 min 100% A; oven: 50° C.; flow rate: 2 ml/min; UV detection: 210 nm.

Method 6B: Instrument: Waters ACQUITY SQD HPLC System; column: Waters Acquity HPLC HSS T3 1.8μ 50 mm×1 mm; eluent A: 1 l of water+0.25 ml of 99% formic acid, eluent B: 1 l of acetonitrile+0.25 ml of 99% formic acid; gradient: 0.0 min 90% A→>1.2 min 5% A→>2.0 min 5% A oven: 50° C.; flow rate: 0.40 ml/min; UV detection: 210-400 nm.

Method 7B: MS instrument type: Waters (Micromass) Quattro Micro; HPLC instrument type: Agilent 1100 Series; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→3.0 min 10%→4.0 min 10% A,→4.01 min 100% A (flow rate 2.5 ml/min)→5.00 min 100% A; oven: 50° C.; flow rate: 2 ml/min; UV detection: 210 nm.

### Preparative Separation of Diastereomers:

Method 1C: Phase: Xbrdge C18, 5 μm OBD 19 mm×150 mm, eluent: acetonitrile/0.1% ammonia solution 55:45; flow rate: 25 ml/min, temperature: 28° C.; UV detection: 210 nm.

### Preparative Separation of Enantiomers:

Method 1D: Phase: Daicel Chiralpak AD-H, 5 μm 250 mm×20 mm, eluent: isohexane/isopropanol 40:60; flow rate: 15 ml/min, temperature: 40° C.; UV detection: 220 nm.

Method 2D: Phase: Daicel Chiralpak AD-H, 5 μm 250 mm×20 mm, eluent: isohexane/ethanol 25:75; flow rate: 15 ml/min, temperature: 40° C.; UV detection: 220 nm.

Method 3D: Phase: Daicel Chiralpak AD-H, 5 μm 250 mm×20 mm; eluent: isopropanol/isohexane 50:50; flow rate: 18 ml/min; temperature: 24° C.; UV detection: 230 nm.

Method 4D: Phase: Daicel Chiralpak AD-H, 5 μm 250 mm×20 mm; eluent: isopropanol/isohexane 50:50; flow rate: 15 ml/min; temperature: 40° C.; UV detection: 220 nm.

Method 5D: Phase: Daicel Chiralpak AD-H, 5 μm 250 mm×20 mm, eluent: ethanol 100%; flow rate: 12 ml/min, temperature: 40° C.; UV detection: 220 nm.

Method 6D: Phase: Daicel Chiralpak AD-H, 5 μm 250 mm×20 mm, eluent: isohexane/ethanol 30:70; flow rate: 15 ml/min, temperature: 40° C.; UV detection: 220 nm.

### Analytical Separation of Enantiomers:

Method 1E: Phase: Daicel Chiralpak AD-H, 5 μm 250 mm×4 mm; eluent: isopropanol/isohexane: 50:50; flow rate: 1 ml/min; temperature: 24° C.; UV detection: 230 nm.

Method 2E: Phase: Daicel Chiralpak AD-H, 5 μm 250 mm×4.6 mm; eluent: isohexane/isopropanol 25:75+0.2% trifluoroacetic acid+1% water; flow rate: 1 ml/min; temperature: 45° C.; UV detection: 235 nm.

Method 3E: Phase: Daicel Chiralpak AD-H, 5 μm 250 mm×4.6 mm; eluent: ethanol 100%; flow rate: 1 ml/min; temperature: 40° C.; UV detection: 220 nm.

Method 4E: Phase: Daicel Chiralpak AS-H, 5 μm 250 mm×4.6 mm; eluent: isohexane/ethanol 30:70; flow rate: 1 ml/min; temperature: 40° C.; UV detection: 220 nm.

The microwave reactor used was a “single mode” instrument of the Emrys™ Optimizer type.

### Starting Compounds

### General Method 1A: Suzuki Coupling

A mixture of the appropriate bromopyridine in toluene (1.8 ml/mmol) is admixed under argon at RT with tetrakis(triphenylphosphine)palladium (0.02 eq.), with a solution of the appropriate arylboronic acid (1.2 eq.) in ethanol (0.5 ml/mmol) and with a solution of potassium fluoride (2.0 eq.) in water (0.2 ml/mmol). The reaction mixture is stirred under reflux for several hours until the conversion is substantially complete. After addition of ethyl acetate and phase separation, the organic phase is washed once with water and once with saturated aqueous sodium chloride solution, dried (magnesium sulphate), filtered and concentrated under reduced pressure. The crude product is purified by flash chromatography (silica gel 60, eluent: dichloromethane/methanol mixtures).

### General Method 2A: Hydrogenation of the Pyridine

A solution of the pyridine in ethanol (9 ml/mmol) is admixed under argon with palladium on activated carbon (moistened with approx. 50% water, 0.3 g/mmol), and the mixture is hydrogenated at 60° C. in a 50 bar hydrogen atmosphere overnight. The catalyst is then filtered off through a filter layer and washed repeatedly with ethanol. The combined filtrates are concentrated under reduced pressure.

General Method 3A: Reaction with Carbamoyl Chlorides or Carbonyl Chlorides

A solution of the piperidine in dichloromethane (2.5 ml/mmol) is admixed dropwise under argon at 0° C. with N,N-diisopropylethylamine (1.2 eq.) and the appropriate carbamoyl chloride or carbonyl chloride (1.2 eq.). The reaction mixture is stirred at RT. After addition of water and phase separation, the organic phase is washed three times with water and once with saturated aqueous sodium chloride solution, dried (sodium sulphate), filtered and concentrated under reduced pressure.

### General Method 4A: Hydrolysis

A solution of the appropriate ester in a mixture of tetrahydrofuran/water (3:1, 12.5 ml/mmol) is admixed at RT with lithium hydroxide (2 eq.). The reaction mixture is stirred at 60° C. and then adjusted to pH 1 with aqueous 1 N hydrochloric acid solution. After adding water/ethyl acetate, the aqueous phase is extracted three times with ethyl acetate. The combined organic phases are dried (sodium sulphate), filtered and concentrated under reduced pressure.

### General Method 5A: N′-Hydroxyimidamide Formation

A solution of the appropriate nitrile (1.0 eq.) in ethanol (1.2 ml/mmol) is admixed at RT with hydroxylammonium chloride (1.5 eq.) and triethylamine (1.2 eq.). The reaction mixture is stirred at room temperature overnight. For workup, ethanol is removed under reduced pressure, and the reaction mixture is admixed with saturated aqueous sodium hydrogencarbonate solution and extracted with ethyl acetate. The organic phase is dried over sodium sulphate and concentrated. The residue is converted without further purification.

### General Method 6A: N′-Hydroxyimidamide Formation

A solution of the appropriate nitrile (1.0 eq.) in a mixture of ethanol (1.9 ml/mmol) and water (0.5 ml/mmol) is admixed at RT with hydroxylammonium chloride (1.08 eq.) and sodium hydroxide (1.12 eq.). The reaction mixture is stirred at room temperature for 16 h. For workup, the reaction mixture is concentrated under reduced pressure, admixed with dichloromethane and filtered. The filtrate is concentrated under reduced pressure and the residue is converted without further purification.

General Method 7A: Reaction with Carbonyl Chlorides

The carboxylic acid is dissolved in dioxane/dimethylformamide (3:1, 1 ml/mmol) and heated to 60° C. After addition of N,N′-carbonyldiimidazole (1.5 eq.), dissolved in dioxane/dimethylformamide (4:1, 1.6 ml/mmol), is stirred at 60° C. for 3 h. After cooling to RT, the carboximidamide, dissolved in 1:1 dioxane/dimethylformamide, is added dropwise and the mixture is stirred at 40° C. overnight. Thereafter, the dioxane is removed under reduced pressure. The residue dissolved in dimethylformamide is subsequently stirred at 115° C. for 1 h. After cooling, the reaction mixture is diluted with water. After extraction with dichloomethane, the organic phase is dried over sodium sulphate and the crude product is purified by means of preparative HPLC.

### General Method 8A: Urea Formation

A solution of the nitrophenyl carbamate (1.0 eq.) in dimethylformamide (10 ml/mmol) is admixed at RT with the appropriate amine (2.0-3.0 eq.) and potassium carbonate (1.0 eq.), and the mixture is stirred in 15 ml portions in a single-mode microwave (Emrys Optimizer) at 150° C. for 0.5-1 h. The reaction solution is filtered and the filtrate is purified by means of preparative HPLC.

### General Method 9A: Methyl Ester Hydrolysis/Epimerization

At RT, potassium tert-butoxide (10 eq.) is added to a solution of the appropriate methyl ester (1.0 eq.) in methanol (35-40 ml/mmol). The mixture is stirred at 60° C. overnight. If the conversion is incomplete, water (1.0 eq.) is added and the mixture is stirred at 60° C. until the conversion is complete. For workup, the methanol is removed under reduced pressure, the residue is admixed with water and the mixture is acidified (pH 1) with aqueous 1 N hydrochloric acid solution. The mixture is extracted with ethyl acetate and the organic phase is dried with magnesium sulphate, filtered and concentrated under reduced pressure.

### Example 1A

### Methyl 5-(4-ethylphenyl)pyridine-3-carboxylate

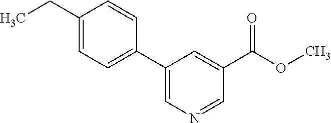

According to General Method 1A, 32 g (148 mmol) of methyl 5-bromonicotinate and 27 g (178 mmol, 1.2 eq.) of 4-ethylphenylboronic acid were reacted. Yield: 24 g (64% of theory)

LC-MS (Method 3B): Rₜ=2.03 min; MS (ESIpos): m/z=242 [M+H]⁺.

### Example 2A

### Methyl 5-(4-ethylphenyl)piperidine-3-carboxylate [racemic cis/trans isomer mixture]

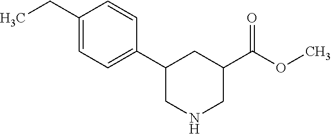

According to General Method 2A, 24 g (94 mmol) of methyl 5-(4-ethylphenyl)pyridine-3-carboxylate were hydrogenated. Yield: 20 g (77% of theory)

LC-MS (Method 5B): Rₜ=1.43 min; MS (ESIpos): m/z=248 [M+H]⁺.

### Example 3A

### Ethyl 5-(4-ethylphenyl)pyridine-3-carboxylate

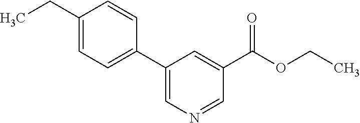

According to General Method 1A, 29 g (126 mmol) of ethyl 5-bromonicotinate and 23 g (152 mmol, 1.2 eq.) of 4-ethylphenylboronic acid were reacted. Yield: 32 g (82% of theory)

LC-MS (Method 4B): Rₜ=3.80 min; MS (ESIpos): m/z=256 [M+H]⁺.

### Example 4A

### Ethyl 5-(4-ethylphenyl)piperidine-3-carboxylate [racemic cis/trans isomer mixture]

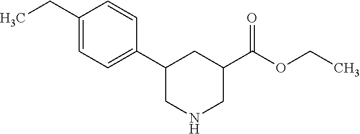

According to General Method 2A, 24 g (71 mmol) of ethyl 5-(4-ethylphenyl)pyridine-3-carboxylate were hydrogenated. Yield: 15 g (81% of theory)

LC-MS (Method 5B): Rₜ=1.78 min and 1.91 min (cis/trans isomers); MS (ESIpos): m/z=262 [M+H]⁺.

### Example 5A

### Ethyl 5-(4-ethylphenyl)piperidine-3-carboxylate [racemic cis isomer]

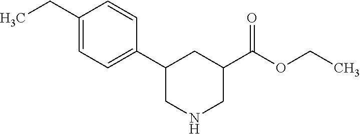

The diastereomer separation of 15 g (124 mmol) of the compound from Example 4A according to Method 1C gave 2.5 g (17% of theory) of the cis isomer (Example 5A).

LC-MS (Method 3B): Rₜ=1.02 min; MS (ESIpos): m/z=262 [M+H]⁺.

### Example 6A

### Ethyl 5-(4-ethylphenyl)piperidine-3-carboxylate [racemic trans isomer]

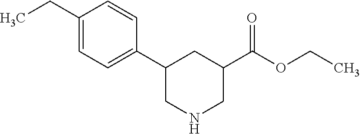

The diastereomer separation of 15 g (124 mmol) of the compound from Example 4A according to Method 1C gave 3.0 g (20% of theory) of the trans isomer (Example 6A).

LC-MS (Method 3B): Rₜ=1.09 min; MS (ESIpos): m/z=262 [M+H]⁺.

### Example 7A

### 3-Ethyl 1-(4-nitrophenyl)5-(4-ethylphenyl)piperidine-1,3-dicarboxylate [racemic cis isomer]

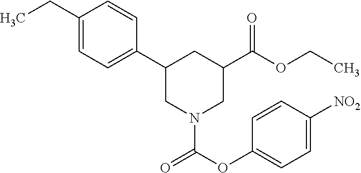

At 0° C., 1.93 g (9.57 mmol) of 4-nitrophenyl chloroformate were added slowly to 2.5 g (9.57 mmol) of ethyl 5-(4-ethylphenyl)piperidine-3-carboxylate (Example 5A) and 1.94 g (19.1 mmol) of triethylamine in 292 ml of dichloromethane. The mixture was stirred at RT for 2 h. For workup, the reaction mixture was washed first with saturated aqueous sodium hydrogencarbonate solution, then with water. The organic phase was dried over sodium sulphate and concentrated under reduced pressure. The residue was purified by means of preparative HPLC.

Yield: 2.66 g (64% of theory)

LC-MS (Method 2B): Rₜ=1.57 min; MS (ESIpos): m/z=427 [M+H]⁺.

### Example 8A

### Ethyl 5-(4-ethylphenyl)1-[(4-hydroxypiperidin-1-yl)carbonyl]piperidine-3-carboxylate [racemic cis isomer]

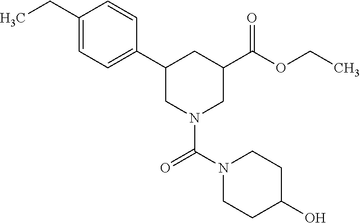

370 mg (0.81 mmol) of 3-ethyl 1-(4-nitrophenyl)5-(4-ethylphenyl)piperidine-1,3-dicarboxylate, 245 mg (2.42 mmol) of 4-hydroxypiperidine and 112 mg (0.81 mmol) of potassium carbonate were added to 9 ml of DMF and heated in a single-mode microwave (Emrys Optimizer) at 150° C. for 15 min. For workup, the reaction solution was admixed with water and extracted with ethyl acetate. The organic phase was dried with sodium sulphate and concentrated under reduced pressure. The residue was purified by preparative HPLC. Yield: 208 mg (66% of theory)

LC-MS (Method 2B): Rₜ=1.23 min; MS (ESIpos): m/z=389 [M+H]⁺.

### Example 9A

### 5-(4-Ethylphenyl)-1-[(4-hydroxypiperidin-1-yl)carbonyl]piperidine-3-carboxylic acid [racemic cis isomer]

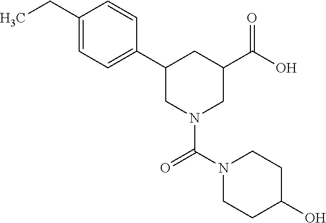

880 mg (2.24 mmol) of ethyl 5-(4-ethylphenyl)-1-[(4-hydroxypiperidin-1-yl)carbonyl]piperidine-3-carboxylate were dissolved in a mixture of 15.5 ml of dioxane and 7.7 ml of water, 215 mg (8.97 mmol) of lithium hydroxide were added and the mixture was stirred at RT overnight. For workup, the reaction solution was concentrated under reduced pressure, then water was added and the mixture was acidified with 1N hydrochloric acid. The precipitate formed was filtered off, washed and dried under reduced pressure. The filtrate was extracted with ethyl acetate. The combined organic phases were dried over sodium sulphate and concentrated under reduced pressure. The two solids gave a total yield of 764 mg (95% of theory).

LC-MS (Method 3B): Rₜ=1.49 min; MS (ESIpos): m/z=361 [M+H]⁺.

### Example 10A

### 3-Methyl 1-(4-nitrophenyl)5-(4-ethylphenyl)piperidine-1,3-dicarboxylate [racemic cis/trans isomer mixture]

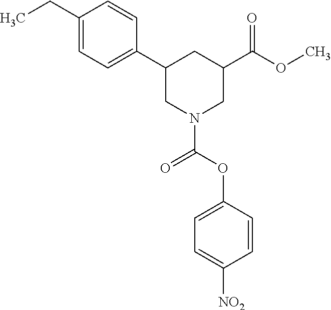

3.0 g (12.1 mmol) of the compound from Example 2A were initially charged in 30 ml of dichloromethane and cooled to 0° C., and admixed with 3.4 ml (2.4 g, 12.1 mmol) of triethylamine and 2.4 g (12.1 mmol) of 4-nitrophenyl chloroformate. The reaction mixture was allowed to warm up slowly to RT and stirred at RT for 16 h. The mixture was washed several times with water, dried over sodium sulphate, filtered and concentrated under reduced pressure. The residue was purified by column chromatography on silica gel (eluent dichloromethane→dichloromethane/methanol 100:2). Yield: 4.7 g (83% of theory, purity 89%)

HPLC (Method 1A): Rₜ=4.94 min and 5.00 min (cis/trans isomer); MS (ESIpos): m/z=413 [M+H]⁺.

### Example 11A

### Methyl 5-(4-ethylphenyl)-1-[(3-hydroxyazetidin-1-yl)carbonyl]piperidine-3-carboxylate [racemic cis/trans isomer mixture]

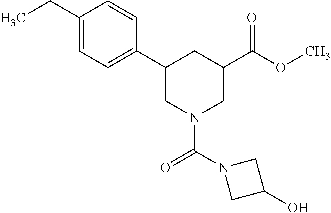

0.3 g (0.7 mmol) of the compound from Example 10A, 0.2 g (2.18 mmol) of 3-hydroxyazetidine hydrochloride and 0.2 g (1.4 mmol) of potassium carbonate were initially charged in 6 ml of DMF and reacted in a single-mode microwave (Emrys Optimizer) at 150° C. for 30 min. The crude product was purified by preparative HPLC. Yield: 105 mg (40% of theory)

LC-MS (Method 3B): Rₜ=1.76 min and 1.85 [cis/trans isomers]; MS (ESIpos): m/z=361 [M+H]⁺.

### Example 12A

### 5-(4-Ethylphenyl)-1-[(3-hydroxyazetidin-1-yl)carbonyl]piperidine-3-carboxylic acid [racemic cis isomer]

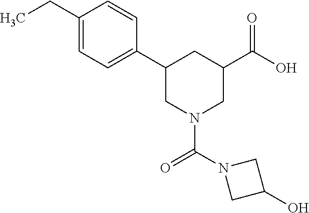

300 mg (0.83 mmol) of the compound from Example 11A were reacted according to General Method 9A. The reaction led selectively to the cis isomer. Yield: 250 mg (90% of theory)

LC-MS (Method 3B): Rₜ=1.44 min; MS (ESIpos): m/z=333 [M+H]⁺;

¹H NMR (400 MHz, DMSO-d₆): δ=12.42 (br s, COOH), 7.18-7.13 (m, 4H), 5.54 (br s, OH), 4.39-4.33 (m, 1H), 4.08-3.97 (m, 3H), 3.68-3.62 (m, 3H), 2.78-2.70 (m, 2H), 2.68-2.54 (m, 3H), 2.48-2.42 (m, 1H), 2.08 (br d, 1H), 1.71 (q, 1H), 1.15 (t, 3H).

### Example 13A

### Methyl 5-[4-(trifluoromethoxy)phenyl]pyridine-3-carboxylate

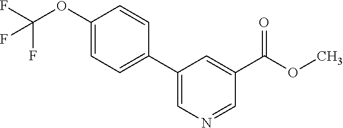

According to General Method 1A, 23 g (105 mmol) of methyl 5-bromonicotinate and 26 g (126 mmol, 1.2 eq.) of 4-trifluoromethoxyphenylboronic acid were reacted. Yield: 14 g (41% of theory)

LC-MS (Method 1B): Rₜ=2.44 min; MS (ESIpos): m/z=298 [M+H]⁺.

### Example 14A

### Methyl 5-[4-(trifluoromethoxy)phenyl]piperidine-3-carboxylate [racemic cis/trans isomer mixture]

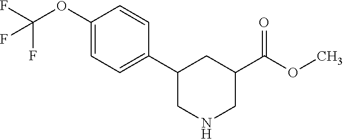

According to General Method 2A, 14 g (45 mmol) of methyl 5-[4-(trifluoromethoxy)phenyl]pyridine-3-carboxylate were hydrogenated. Yield: 8 g (59% of theory)

LC-MS (Method 1B): Rₜ=1.29 min and 1.33 min (cis/trans isomers); MS (ESIpos): m/z=304 [M+H]⁺.

### Example 15A

### 3-Methyl 1-(4-nitrophenyl)5-[4-(trifluoromethoxy)phenyl]piperidine-1,3-dicarboxylate [racemic cis/trans isomer mixture]

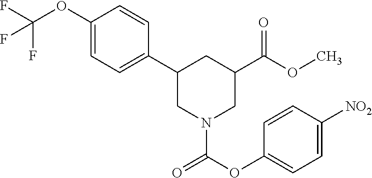

At 0° C., 5.32 g (26.4 mmol) of 4-nitrophenyl chloroformate were added slowly to 8.0 g (26.4 mmol) of methyl 5-(4-(trifluoromethoxy)phenyl)piperidine-3-carboxylate (Example 15A) and 5.34 g (26.3 mmol) of triethylamine in 666 ml of dichloromethane. The mixture was stirred at RT for 2 h. For workup, the reaction mixture was washed first with saturated aqueous sodium hydrogencarbonate solution, then with water. The organic phase was dried over sodium sulphate and concentrated under reduced pressure. The residue was purified by means of flash chromatography on silica gel (eluent: cyclohexane/ethyl acetate 1:2 to 1:1). Yield: 7.32 g (54% of theory)

LC-MS (Method 3B): Rₜ=2.47 min; MS (ESIpos): m/z=469 [M+H]⁺.

### Example 16A

### Methyl 1-[(4-hydroxypiperidin-1-yl)carbonyl]-5-[4-(trifluoromethoxy)phenyl]piperidine-3-carboxylate [racemic cis/trans isomer mixture]

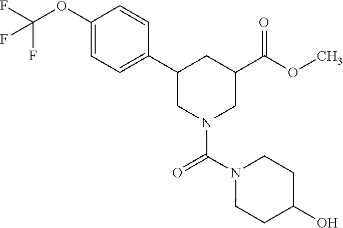

1780 mg (3.80 mmol) of 3-methyl 1-(4-nitrophenyl)-5-[4-trifluoromethoxy)phenyl]piperidine-1,3-dicarboxylate, 1153 mg (11.40 mmol) of 4-hydroxypiperidine and 525 mg (3.80 mmol) of potassium carbonate were added to 37 ml of DMF and heated in 2 portions in a single mode microwave (Emrys Optimizer) at 150° C. for 15 min. For workup, the two reaction solutions were combined, admixed with water and extracted with ethyl acetate. The organic phase was dried with sodium sulphate and concentrated under reduced pressure. The residue was purified by means of preparative HPLC. Yield: 849 mg (50% of theory).

LC-MS (Method 2B): Rₜ=1.23 min; MS (ESIpos): m/z=431 [M+H]⁺.

### Example 17A

### 1-[(4-Hydroxypiperidin-1-yl]carbonyl]-5-[4-(trifluoromethoxy)phenyl]piperidine-3-carboxylic acid [racemic cis isomer]

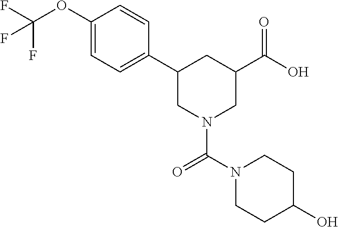

828 mg (1.92 mmol) of methyl 5-(4-trifluoromethoxy)phenyl)-1-[(4-hydroxypiperidin-1-yl)carbonyl]piperidine-3-carboxylate were dissolved in 70 ml of methanol, admixed with 2159 mg (19.24 mmol) of potassium tert-butoxide and stirred at 60° C. overnight. For workup, the reaction solution was diluted with water and acidified with 1N hydrochloric acid (pH 1). The mixture was extracted with ethyl acetate. The combined organic phases were dried over sodium sulphate and concentrated under reduced pressure. The reaction led selectively to the cis isomer. Yield: 749 mg (94% of theory).

LC-MS (Method 2B): Rₜ=1.04 min; MS (ESIpos): m/z=417 [M+H]⁺.

### Example 18A

### Methyl 1-[(3-hydroxyazetidin-1-yl)carbonyl]-5-[4-(trifluoromethoxy)phenyl]piperidine-3-carboxylate [racemic cis/trans isomer mixture]

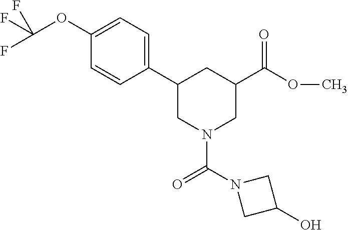

According to General Method 8A, 300 mg (0.38 mmol) of the compound from Example 15A and 89 mg (1.15 mmol) of 3-hydroxyazetidine were converted. Yield: 100 mg (63% of theory).

LC-MS (Method 6B): Rₜ=0.97 min and 0.99 min (cis/trans isomers); MS (ESIpos): m/z=403 [M+H]⁺.

### Example 19A

### 1-[(3-Hydroxyazetidin-1-yl)carbonyl]-5-[4-(trifluoromethoxy)phenyl]piperidine-3-carboxylic acid [racemic cis isomer]

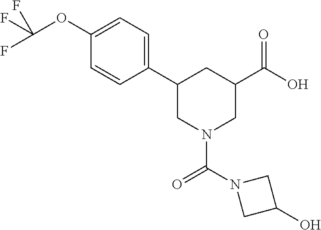

According to General Method 9A, 700 mg (1.43 mmol) of the compound from Example 18A and 1.61 g (14.3 mmol) of potassium tert-butoxide were converted. The reaction led selectively to the cis isomer. Yield: 700 g (99% of theory, 84% purity).

LC-MS (Method 6B): Rₜ=0.87 min; MS (ESIpos): m/z=389 [M+H]⁺.

### Example 20A

### Methyl 5-[4-(trifluoromethyl)phenyl]pyridine-3-carboxylate

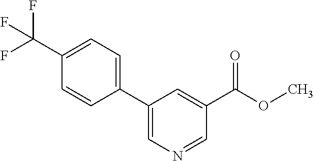

According to General Method 1A, 28 g (132 mmol) of methyl 5-bromonicotinate and 30 g (158 mmol, 1.2 eq.) of 4-trifluoromethylphenylboronic acid were reacted. Yield: 32 g (85% of theory)

LC-MS (Method 5B): Rₜ=2.27 min; MS (ESIpos): m/z=282 [M+H]⁺.

### Example 21A

### Methyl 5-[4-(trifluoromethyl)phenyl]piperidine-3-carboxylate [racemic cis/trans isomer mixture]

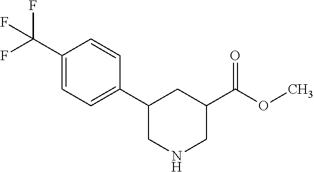

According to General Method 2A, 32 g (112 mmol) of methyl 5-[4-(trifluoromethyl)phenyl]pyridine-3-carboxylate (Example 20A) were hydrogenated. Yield: 26 g (82% of theory)

LC-MS (Method 1B): Rₜ=1.35 and 1.41 min (cis/trans isomers); MS (ESIpos): m/z=288 [M+H]⁺.

### Example 22A

### 3-Methyl 1-(4-nitrophenyl)5-[4-(trifluoromethyl)phenyl]piperidine-1,3-dicarboxylate [racemic cis/trans isomer mixture]

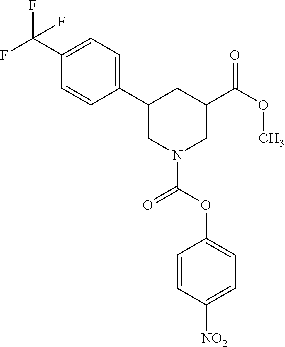

20.0 g (69.6 mmol) of methyl 5-[4-(trifluoromethyl)phenyl]piperidine-3-carboxylate (Example 21A) were dissolved in 1.0 l of dichloromethane and admixed at 0° C. with 14.1 g (139 mmol) of triethylamine. Subsequently, 14.0 g (69.6 mmol) of 4-nitrophenyl chlorocarbonate were added dropwise. The reaction mixture was stirred at 0° C. for 2 h and then at RT for 16 h. For workup, the mixture was washed with saturated aqueous sodium hydrogencarbonate solution. The organic phase was dried over magnesium sulphate, filtered and concentrated under reduced pressure. This gave 31.3 g of crude product, which was reacted without any further purification steps.

LC-MS (Method 3B): Rₜ=2.44 min and 2.48 min (cis/trans isomers); MS (ESIpos): m/z=453 [M+H]⁺.

### Example 23A

### Methyl 1-[(4-hydroxypiperidin-1-yl)carbonyl]-5-[4-(trifluoromethyl)phenyl]piperidine-3-carboxylate [racemic cis/trans isomer mixture]

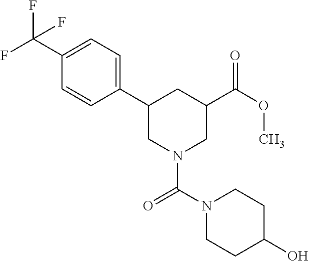

According to General Method 8A, 4.00 g (8.84 mmol) of 3-methyl 1-(4-nitrophenyl)-5-[4-(trifluoromethyl)phenyl]piperidine-1,3-dicarboxylate (Example 22A) and 2.68 g (26.5 mmol) of 4-hydroxypiperidine were converted. Yield: 3.10 g (83% of theory).

LS-MC (Method 3B): Rₜ=2.72 min and 2.78 min (cis/trans isomers); MS (ESIpos): m/z=415 [M+H]⁺.

¹H NMR (400 MHz, DMSO-d₆): δ=7.69 (d, 2H), 7.53 (t, 2H), 4.67 (br d, 1H), 3.91-3.78 (m, 1H), 3.66-3.34 (m, 7H), 3.15-3.05 (m, 1H), 2.96-2.65 (m, 5H), 2.25-2.11 (m, 1H), 1.96-1.63 (m, 3H), 1.38-1.18 (m, 2H).

### Example 24A

### 1-[(4-hydroxypiperidin-1-yl)carbonyl]-5-[4-(trifluoromethyl)phenyl]piperidine-3-carboxylic acid [racemic cis isomer]

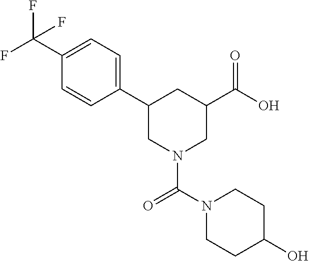

According to General Method 9A, 2.10 g (5.07 mmol) of methyl 1-[(4-hydroxypiperidin-1-yl)carbonyl]-5-[4-(trifluoromethyl)phenyl]piperidine-3-carboxylate (Example 23A) were converted. The reaction led selectively to the cis isomer. Yield: 2.02 g (99% of theory).

LC-MS (Method 2B): Rₜ=1.01 min; MS (ESIpos): m/z=401 [M+H]⁺.

### Example 25A

### Methyl 1-[(3-hydroxyazetidin-1-yl)carbonyl]-5-[4-(trifluoromethyl)phenyl]piperidine-3-carboxylate [racemic cis/trans isomer mixture]

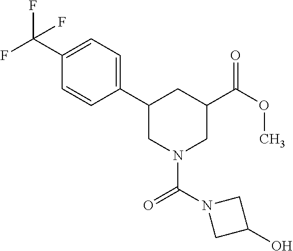

According to General Method 8A, 4.00 g (8.84 mmol) of 3-methyl 1-(4-nitrophenyl)-5-[4-(trifluoromethyl)phenyl]piperidine-1,3-dicarboxylate (Example 22A), 2.91 g (26.5 mmol) of 4-azetidin-3-ol hydrochloride and potassium carbonate (2.5 eq.) were converted. Yield: 2.48 g (70% of theory).

LC-MS (Method 2B): Rₜ=1.08 min and 1.10 min (cis/trans isomers); MS (ESIpos): m/z=387 [M+H]⁺.

### Example 26A

### 1-[(3-Hydroxyazetidin-1-yl)carbonyl]-5-[4-(trifluoromethyl)phenyl]piperidine-3-carboxylic acid [racemic cis isomer]

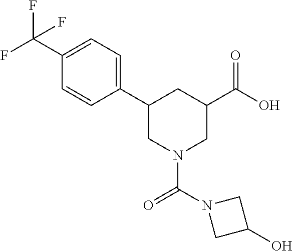

According to General Method 9A (reaction time: 2 h), 2.48 g (6.42 mmol) of methyl 1-[(3-hydroxyazetidin-1-yl)carbonyl]-5-[4-(trifluoromethyl)phenyl]piperidine-3-carboxylate (Example 25A) were converted. The reaction led selectively to the cis isomer. Yield: 2.33 g (94% of theory).

LS-MS (Method 1B): Rₜ=1.84 min; MS (ESIpos): m/z=373 [M+H]⁺.

¹H NMR (400 MHz, DMSO-d₆): δ=12.4 (br s, 1H), 7.69 (d, 2H), 7.53 (d, 2H), 5.57 (d, 1H), 4.42-4.32 (m, 1H), 4.11-3.95 (m, 3H), 3.77-3.63 (m, 3H), 3.32 (hidden, 1H), 2.88-2.76 (m, 3H), 3.14 (br d, 1H), 1.85-1.72 (m, 1H).

### Example 27A

### 1-tert-butyl 3-methyl-5-[4-(trifluoromethoxy)phenyl]piperidine-1,3-dicarboxylate [racemic cis/trans isomer mixture]

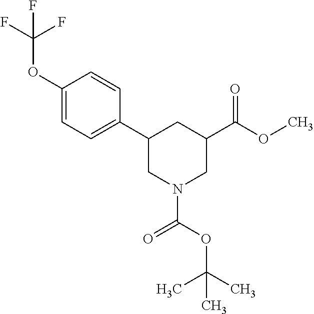

1.0 g (3.40 mmol) of the compound from Example 14A and 0.47 ml (0.34 g, 3.40 mmol) of triethylamine were initially charged in 50 ml of dichloromethane and admixed at RT with 0.78 ml (0.74 g, 3.40 mmol) of di-tert-butyl dicarboxylate. The reaction mixture was stirred at RT for 16 h, washed with water, dried over sodium sulfate, filtered and concentrated under reduced pressure. The residue was purified by column chromatography on silica gel (eluent: 50:1 to 20:1 dichloromethane/methanol). Yield: 1.32 g (78% of theory, 81% purity).

LC-MS (Method 5B): Rₜ=2.66 min and 2.72 min [cis/trans isomers]; MS (ESIpos): m/z=404 [M+H]⁺.

### Example 28A

### 1-(tert-butoxycarbonyl)-5-[4-(trifluoromethoxy)phenyl]piperidine-3-carboxylic acid [racemic cis isomer]

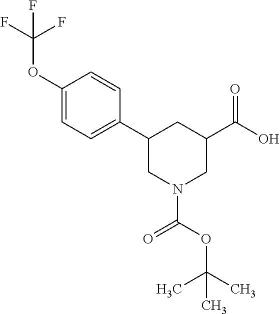

1.3 g (3.27 mmol) of the compound from Example 27A were converted according to General Method 9A. The reaction led selectively to the cis isomer. Yield: 1.31 g (87% of theory, 85% purity).

LC-MS (Method 5B): Rₜ=2.46 min; MS (ESIpos): m/z=390 [M+H]⁺.

### Example 29A

### tert-Butyl 3-(3-cyclopropyl-1,2,4-oxadiazol-5-yl)-5-[4-(trifluoromethoxy)phenyl]piperidine-1-carboxylate [racemic cis isomer]

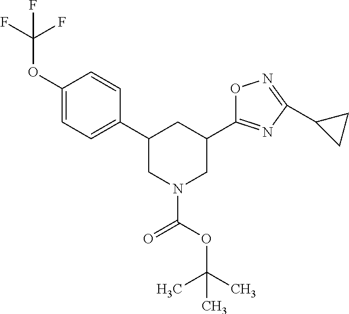

According to General Method 7A, 2.37 g (5.05 mmol) of 1-(tert-butoxycarbonyl)-5-[4-(trifluoromethoxy)phenyl]piperidine-3-carboxylic acid (Example 28A) and 0.76 g (7.58 mmol, 1.5 eq.) of N′-hydroxycyclopropanecarboximid amide were converted. 2.46 g of crude product were obtained, which were converted without further purifying operations.

LC-MS (Method 6B): Rₜ=1.44 min; MS (ESIpos): m/z=454 [M+H]⁺.

### Example 30A

### 3-(3-Cyclopropyl-1,2,4-oxadiazol-5-yl)-5-[4-(trifluoromethoxy)phenyl]piperidine trifluoroacetate [racemic cis isomer]

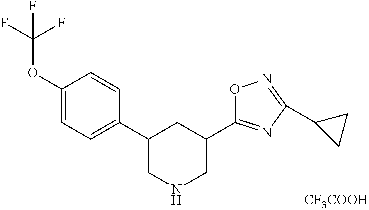

To a solution of 3.0 g (approx. 6.62 mmol) of the compound from Example 29A in 405 ml of dichloromethane were added 7.50 ml (99.2 mmol) of trifluoroacetic acid, and the mixture was stirred at room temperature for 20 h. Subsequently, the reaction mixture was concentrated under reduced pressure, admixed with dichloromethane and concentrated again. 3.0 g of crude product were obtained, which were converted without further purifying operations.

LC-MS (Method 6B): Rₜ=0.85 min; MS (ESIpos): m/z=354 [M+H-TFA]⁺.

### Example 31A

### 4-Nitrophenyl 3-(3-cyclopropyl-1,2,4-oxadiazol-5-yl)-5-[4-(trifluoromethoxy)phenyl]piperidine-1-carboxylate [racemic cis isomer]

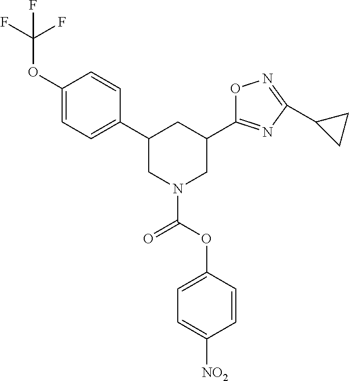

3.0 g (approx. 6.42 mmol) of 3-(3-cyclopropyl-1,2,4-oxadiazol-5-yl)-5-[4-(trifluoromethoxy)phenyl]piperidine trifluoroacetate (Example 30A) were dissolved in 122 ml of dichloromethane and admixed at 0° C. with 2.60 g (25.6 mmol) of triethylamine. Subsequently, 1.29 g (6.42 mmol) of 4-nitrophenyl chlorocarbonate were added dropwise. The reaction mixture was stirred at 0° C. for 2 h and then at RT for 16 h. For workup, the mixture was washed with saturated aqueous sodium hydrogencarbonate solution. The organic phase was dried over magnesium sulfate, filtered and concentrated under reduced pressure. 3.19 g of crude product were obtained, which were converted without further purifying operations.

LC-MS (Method 6B): Rₜ=1.38 min; MS (ESIpos): m/z=519 [M+H]⁺.

### Example 32A

### N′-Hydroxy-3-methoxypropanimid amide

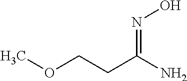

According to General Method 6A, 20.0 g (235.0 mmol) of 3-methoxypropionitrile were converted.

Yield: 18.1 g (49% of theory, 74% purity).

HPLC (Method 1A): Rₜ=0.35 min; MS (ESIpos): m/z=119 [M+H]⁺.

### Example 33A

### 3-Ethoxy-N′-hydroxypropanimid amide

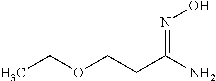

According to General Method 5A, 5.0 g (50.4 mmol) of 3-ethoxypropionitrile were converted.

Yield: 0.6 g (8% of theory, 90% purity)

HPLC (Method 1A): Rₜ=0.60 min; MS (ESIpos): m/z=133 [M+H]⁺.

### Example 34A

### N′-Hydroxycyclopropanecarboximid amide

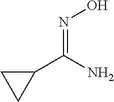

According to General Method 5A, 7.2 g (107.3 mmol) of cyclopropanecarbonitrile were converted.

Yield: 4.8 g (44% of theory).

LC-MS (Method 2B): Rₜ=0.16 min; MS (ESIpos): m/z=101 [M+H]⁺.

### WORKING EXAMPLES

### General Method 1

### Oxadiazole Formation

A solution of the appropriate piperidine-3-carboxylic acid in dimethylformamide (10 ml/mmol) is admixed under argon at RT with HATU (1.2 eq.), N,N-diisopropylethylamine (2.2 eq.) and the appropriate N′-hydroxyimidamide (1.1 eq.). The reaction mixture is stirred at RT until the formation of the intermediate is complete and then stirred further at 120° C. until the desired product is formed from this intermediate. The reaction mixture is then purified by means of preparative HPLC.

### General Method 2

### Oxadiazole Formation

The carboxylic acid is dissolved in dioxane/dimethylformamide (3:1, 1 ml/mmol) and heated to 60° C. After addition of N,N′-carbonyldiimidazole (1.5 eq.), dissolved in dioxane/dimethylformamide (4:1, 1.6 ml/mmol), the mixture is stirred at 60° C. for 3 h. After cooling to RT, the carboximidamide, dissolved in dioxane/dimethylformamide 1:1, is added dropwise and stirred at 40° C. overnight. The dioxane is then removed under reduced pressure. The residue dissolved in dimethylformamide is then stirred at 115° C. for 1 h. After cooling, the reaction mixture is diluted with water. After extraction with dichloromethane, the organic phase is dried over sodium sulphate and the crude product is purified by means of preparative HPLC.

### General Method 3

### Oxadiazole Formation

A solution of the appropriate piperidine-3-carboxylic acid in dimethylformamide (10 ml/mmol) is admixed under Argon at RT with HATU (1.2 eq.), N,N-diisopropylethylamine (2.2 eq.) and the appropriate N′-hydroxyimidamide (1.1 eq.). The reaction mixture is stirred at RT until complete formation of the intermediate. The flask contents are admixed with ethyl acetate. The organic phase is washed with 1N sodium hydroxide solution, water and saturated aqueous sodium chloride solution. Subsequently, the organic phase is dried over sodium sulphate, filtered and concentrated under reduced pressure. The reaction mixture is purified by means of preparative HPLC. The product fractions are concentrated under reduced pressure, dissolved in DMF and converted in a microwave at 180° C. for 3 minutes. The flask contents are admixed with ethyl acetate. The organic phase is washed with water and saturated aqueous sodium chloride solution. Subsequently, the organic phase is dried over sodium sulphate, filtered and concentrated under reduced pressure. The reaction mixture is purified by means of preparative HPLC.

### Example 1

### {3-[3-(2-Ethoxyethyl)-1,2,4-oxadiazol-5-yl]-5-[4-(trifluoromethoxy)phenyl]piperidin-1-yl}(3-hydroxyazetidin-1-yl)methanone [racemic cis isomer]

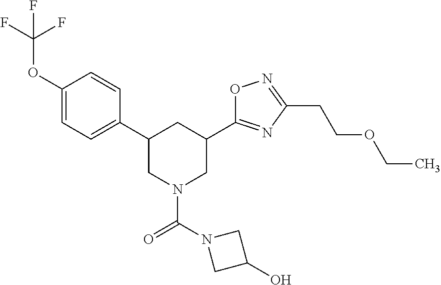

According to General Method 3, 300 mg (0.7 mmol) of the compound from Example 19A and 191 mg (1.4 mmol, 2.0 eq.) of the compound from Example 33A were converted. Yield: 82 mg (22% of theory).

HPLC (Method 6B): Rₜ=1.04 min; MS (ESIpos): m/z=485 [M+H]⁺;

¹H NMR (500 MHz, DMSO-d₆): δ=7.51-7.42 (m, 2H), 7.33 (d, 2H), 5.62-5.50 (m, 1H), 4.43-4.32 (m, 1H), 4.20-4.04 (m, 3H), 3.79-3.60 (m, 5H), 3.43 (q, 2H), 3.07-2.84 (m, 5H), 2.35-2.24 (m, 2H), 1.99 (q, 1H), 1.07 (t, 3H).

### Example 2

### {3-[3-(2-Ethoxyethyl)-1,2,4-oxadiazol-5-yl]-5-[4-(trifluoromethoxy)phenyl]piperidin-1-yl}(3-hydroxyazetidin-1-yl)methanone [enantiomerically pure cis isomer]

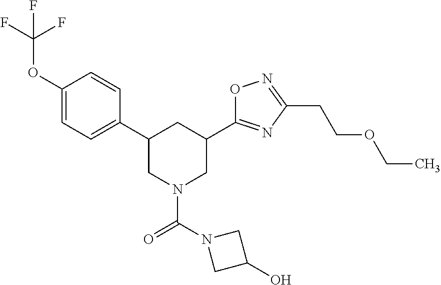

Enantiomer separation of 70 mg of the racemate from Example 1 by Method 1D gave 23 mg of the title compound from Example 2 (enantiomer 1) and 29 mg of the title compound from Example 3 (enantiomer 2).

HPLC (Method 1E): Rₜ=4.94 min, >99.5% ee; MS (ESIpos): m/z=485 [M+H]⁺.

### Example 3

### {3-[3-(2-Ethoxyethyl)-1,2,4-oxadiazol-5-yl]-5-[4-(trifluoromethoxy)phenyl]piperidin-1-yl}(3-hydroyazetidin-1-yl)methanone [enantiomerically pure cis isomer]

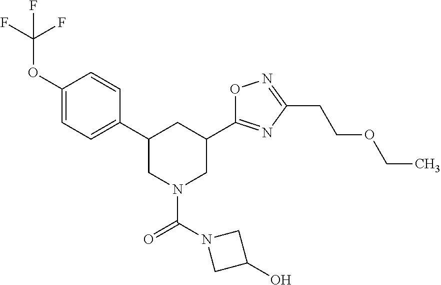

Enantiomer separation of 70 mg of the racemate from Example 1 by Method 1D gave 23 mg of the title compound from Example 2 (enantiomer 1) and 29 mg of the title compound from Example 3 (enantiomer 2).

HPLC (Method 1E): Rₜ=6.14 min, >99.5% ee; MS (ESIpos): m/z=485 [M+H]⁺.

### Example 4

### {3-(3-Cyclopropyl-1,2,4-oxadiazol-5-yl)-5-[4-(trifluoromethyl)phenyl]piperidin-1-yl}(3-hydroxyazetidin-1-yl)methanone [enantiomerically pure cis isomer]

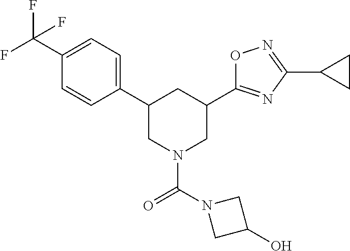

According to General Method 1, 200 mg (0.489 mmol) of the carboxylic acid from Example 26A and 53.8 mg (0.538 mmol) of N′-hydroxycyclopropanecarboximidamide from Example 34A were converted. The crude product was subsequently purified by means of preparative HPLC. Enantiomer separation of the racemate by Method 4D gave 28 mg of the title compound from Example 4 and 30 mg of the title compound from Example 5.

HPLC (Method 2E): Rₜ=4.53 min, >99.0% ee;

LC-MS (Method 6B): Rₜ=1.04 min; MS (ESIpos): m/z=437 [M+H]⁺;

¹H NMR (400 MHz, DMSO-d₆): δ=7.70 (d, 2H), 7.56 (d, 2H), 5.58 (d, 1H), 4.38 (d, 1H), 4.20-4.03 (m, 3H), 3.83-3.63 (m, 3H), 3.30-3.20 (m, 2H), 3.05-2.87 (m, 3H), 2.28 (d, 1H), 2.17-2.07 (m, 1H), 2.06-1.93 (m, 1H), 1.14-0.98 (m, 2H), 0.89 (br s, 2H).

### Example 5

### {3-(3-Cyclopropyl-1,2,4-oxadiazol-5-yl)-5-[4-(trifluoromethyl)phenyl]piperidin-1-yl}(3-hydroxyazetidin-1-yl)methanone [enantiomerically pure cis isomer]

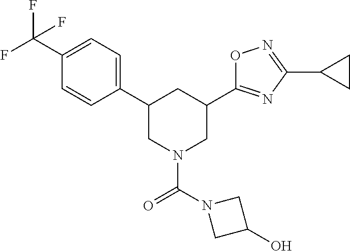

According to General Method 1, 200 mg (0.489 mmol) of the carboxylic acid from Example 26A and 53.8 mg (0.538 mmol) of N′-hydroxycyclopropanecarboximidamide from Example 34A were converted. The crude product was subsequently purified by means of preparative HPLC. Enantiomer separation of the racemate by Method 4D gave 28 mg of the title compound from Example 4 and 30 mg of the title compound from Example 5.

HPLC (Method 2E): Rₜ=5.74 min, >99.0% ee;

LC-MS (Method 6B): Rₜ=1.04 min; MS (ESIpos): m/z=437 [M+H]⁺;

¹H NMR (400 MHz, DMSO-d₆): δ=7.70 (d, 2H), 7.56 (d, 2H), 5.58 (d, 1H), 4.38 (d, 1H), 4.20-4.03 (m, 3H), 3.83-3.63 (m, 3H), 3.30-3.20 (m, 2H), 3.05-2.87 (m, 3H), 2.28 (d, 1H), 2.17-2.07 (m, 1H), 2.06-1.93 (m, 1H), 1.14-0.98 (m, 2H), 0.89 (br s, 2H).

### Example 6

### {3-(3-Cyclopropyl-1,2,4-oxadiazol-5-yl)-5-[4-(trifluoromethoxy)phenyl]piperidin-1-yl}(3-hydroxyazetidin-1-yl)methanone [racemic cis isomer]

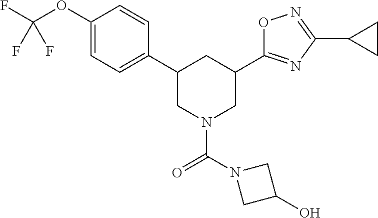

According to General Method 8A, 2.55 g (approx. 3.69 mmol) of 4-nitrophenyl 3-(3-cyclopropyl-1,2,4-oxadiazol-5-yl)-5-[4-(trifluoromethoxy)phenyl]piperidine-1-carboxylate (Example 31A), 1.23 g (11.07 mmol) of 4-azetidin-3-ol hydrochloride and potassium carbonate (2.0 eq.) were converted. Yield: 850 mg (51% of theory).

LC-MS (Method 6B): Rₜ=1.09 min; MS (ESIpos): m/z=453 [M+H]⁺;

¹H NMR (400 MHz, DMSO-d₆): δ=7.45 (d, 2H), 7.35 (d, 2H), 5.52 (d, 1H), 4.42-4.33 (m, 1H), 4.15-4.04 (m, 3H), 3.76-3.65 (m, 3H), 3.24 (ft, 1H), 3.01-2.82 (m, 3H), 2.25 (dm, 1H), 2.15-2.07 (m, 1H), 1.95 (q, 1H); 1.08-1.02 (m, 2H), 0.90-0.86 (m, 2H).

### Example 7

### {3-(3-Cyclopropyl-1,2,4-oxadiazol-5-yl)-5-[4-(trifluoromethoxy)phenyl]piperidin-1-yl}(3-hydroxyazetidin-1-yl)methanone [enantiomerically pure cis isomer]

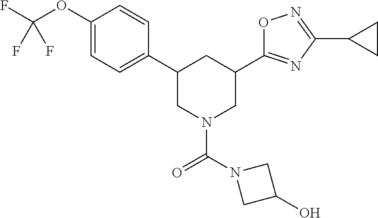

Enantiomer separation of 850 mg of the racemate from Example 6 by Method 6D gave 480 mg of the title compound from Example 7 and 538 mg of the title compound from Example 8.

HPLC (Method 4E): Rₜ=4.94 min, >99.5% ee;

LC-MS (Method 6B): Rₜ=1.09 min; MS (ESIpos): m/z=453 [M+H]⁺;

¹H NMR (400 MHz, DMSO-d₆): δ=7.44 (d, 2H), 7.32 (d, 2H), 5.59 (bs, 1H), 4.41-4.35 (m, 1H), 4.13-4.04 (m, 3H), 3.75-3.65 (m, 3H), 3.01-2.85 (m, 3H), 2.25 (dm, 1H), 2.14-2.08 (m, 1H), 1.95 (q, 1H); 1.09-1.01 (m, 2H), 0.92-0.85 (m, 2H).

### Example 8

### {3-(3-Cyclopropyl-1,2,4-oxadiazol-5-yl)-5-[4-(trifluoromethoxy)phenyl]piperidin-1-yl}(3-hydroxyazetidin-1-yl)methanone [enantiomerically pure cis isomer]

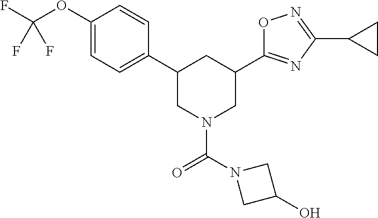

Enantiomer separation of 850 mg of the racemate from Example 6 by Method 6D gave 480 mg of the title compound from Example 7 and 538 mg of the title compound from Example 8.

HPLC (Method 4E): Rₜ=11.89 min, >99.5% ee;

LC-MS (Method 6B): Rₜ=1.09 min; MS (ESIpos): m/z=453 [M+H]⁺;

¹H NMR (400 MHz, DMSO-d₆): δ=7.44 (d, 2H), 7.32 (d, 2H), 5.59 (bs, 1H), 4.41-4.35 (m, 1H), 4.13-4.04 (m, 3H), 3.75-3.65 (m, 3H), 3.01-2.85 (m, 3H), 2.25 (dm, 1H), 2.14-2.08 (m, 1H), 1.95 (q, 1H); 1.09-1.01 (m, 2H), 0.92-0.85 (m, 2H).

### Example 9

### (3-Hydroxyazetidin-1-yl){3-[3-(2-methoxyethyl)-1,2,4-oxadiazol-5-yl]-5-[4-(trifluoromethoxy)-phenyl]piperidin-1-yl}methanone [enantiomerically pure cis isomer]

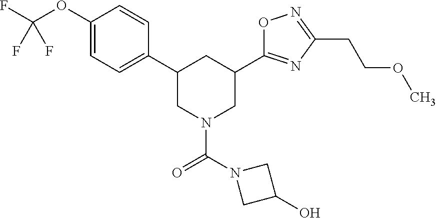

According to General Method 2, 150 mg (0.324 mmol) of the compound from Example 19A and 77 mg (0.649 mmol) of N′-hydroxy-3-methoxypropanimidamide were converted. Yield: 23 mg (15% of theory). Enantiomer separation of 1.06 g of the racemate by Method 5D gave 716 mg of the title compound from Example 9 and 675 mg of the title compound from Example 10.

HPLC (Method 3E): Rₜ=5.88 min, >99.5% ee;

LC-MS (Method 6B): Rₜ=1.01 min; MS (ESIpos): m/z=441 [M+H]⁺;

¹H NMR (400 MHz, DMSO-d₆): δ=7.46 (d, 2H), 7.33 (d, 2H), 5.56 (d, 1H), 4.42-4.35 (m, 1H), 4.20-4.05 (m, 3H), 3.77-3.65 (m, 5H), 3.23 (s, 3H), 3.05-2.85 (m, 5H), 2.30 (dm, 1H), 1.99 (q, 1H).

### Example 10

### (3-Hydroxyazetidin-1-yl){3-[3-(2-methoxyethyl)-1,2,4-oxadiazol-5-yl]-5-[4-(trifluoromethoxy)-phenyl]piperidin-1-yl}methanone [enantiomerically pure cis isomer]

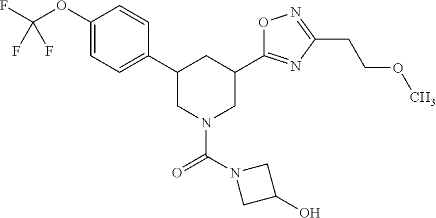

According to General Method 2, 150 mg (0.324 mmol) of the compound from Example 19A and 77 mg (0.649 mmol) of N′hydroxy-3-methoxypropanimidamide were converted. Yield: 23 mg (15% of theory). Enantiomer separation of 1.06 g of the racemate by Method 5D gave 716 mg of the title compound from Example 9 and 675 mg of the title compound from Example 10.

HPLC (Method 3E): Rₜ=12.83 min, >99.5% ee;

LC-MS (Method 6B): Rₜ=1.01 min; MS (ESIpos): m/z=441 [M+H]⁺;

¹H NMR (400 MHz, DMSO-d₆): δ=7.46 (d, 2H), 7.33 (d, 2H), 5.56 (d, 1H), 4.42-4.35 (m, 1H), 4.20-4.05 (m, 3H), 3.77-3.65 (m, 5H), 3.23 (s, 3H), 3.05-2.85 (m, 5H), 2.30 (dm, 1H), 1.99 (q, 1H).

### Example 11

### {3-[3-(2-Ethoxyethyl)-1,2,4-oxadiazol-5-yl]-5-[4-(trifluoromethyl)phenyl]piperidin-1-yl}(3-hydroxyazetidin-1-yl)methanone [enantiomerically pure cis isomer]

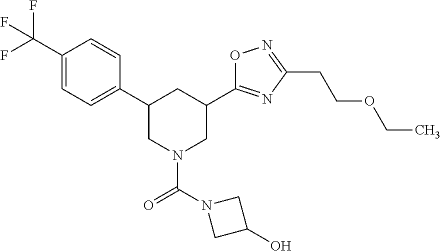

According to General Method 2, 300 mg (0.81 mmol) of the compound from Example 26A and 173 mg (0.121 mmol, 1.5 eq.) of the compound from Example 33A were converted. Enantiomer separation of 133 mg of the racemate by Method 3D gave 61 mg of the title compound from Example 11 (enantiomer 1) and 60 mg of the title compound from Example 12 (enantiomer 2).

HPLC (Method 6B): Rₜ=1.04 min; MS (ESIpos): m/z=469 [M+H]⁺;

HPLC (Method 1E): Rₜ=5.23 min, >99.5% ee;

¹H NMR (500 MHz, DMSO-d₆): δ=7.71 (d, 2H), 7.56 (d, 2H), 5.59 (d, 1H), 4.38 (s, 1H), 4.16 (d, 1H), 4.09 (q, 2H), 3.80-3.65 (m, 5H), 3.43 (q, 2H), 3.09-2.95 (m, 3H), 2.93 (t, 2H), 2.36-2.27 (m, 1H), 2.10-1.96 (m, 1H), 1.13-1.01 (m, 3H).

### Example 12

### {3-[3-(2-Ethoxyethyl)-1,2,4-oxadiazol-5-yl]-5-[4-(trifluoromethyl)phenyl]piperidin-1-yl}(3-hydroxyazetidin-1-yl)methanone [enantiomerically pure cis isomer]

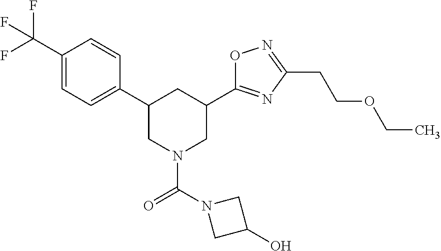

According to General Method 2, 300 mg (0.81 mmol) of the compound from Example 26A and 173 mg (0.121 mmol, 1.5 eq.) of the compound from Example 33A were converted. Enantiomer separation of 133 mg of the racemate by Method 3D gave 61 mg of the title compound from Example 11 (enantiomer 1) and 60 mg of the title compound from Example 12 (enantiomer 2).

HPLC (Method 1E): Rₜ=8.20 min, >99.5% ee; MS (ESIpos): m/z=497 [M+H]⁺.

### Example 13

### {3-[3-(2-Ethoxyethyl)-1,2,4-oxadiazol-5-yl]-5-(4-ethylphenyl)piperidin-1-yl}(4-hydroxypiperidin-1-yl)methanone [racemic cis isomer]

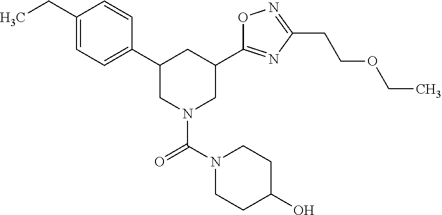

According to General Method 3, 360 mg (1.0 mmol) of the compound from Example 9A and 264 mg (2.0 mmol, 2.0 eq.) of the compound from Example 33A were converted. Yield: 113 mg (25% of theory).

HPLC (Method 6B): Rₜ=1.05 min; MS (ESIpos): m/z=457 [M+H]⁺;

¹H NMR (500 MHz, DMSO-d₆): δ=7.32-7.07 (m, 4H), 4.73-4.58 (m, 1H), 4.00-3.88 (m, 1H), 3.71 (t, 2H), 3.61 (dt, 1H), 3.56-3.37 (m, 6H), 3.17 (d, 1H), 3.03-2.76 (m, 4H), 2.60 (q, 2H), 2.34-2.24 (m, 2H), 2.04-1.87 (m, 1H), 1.81-1.64 (m, 2H), 1.38-1.24 (m, 2H), 1.16 (t, 3H), 1.10-1.02 (m, 3H).

### Example 14

### {3-[3-(2-Ethoxyethyl)-1,2,4-oxadiazol-5-yl]-5-(4-ethylphenyl)piperidin-1-yl}(4-hydroxypiperidin-1-yl)methanone [enantiomerically pure cis isomer]

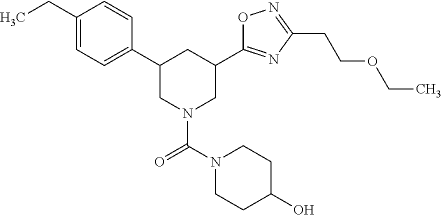

Enantiomer separation of 113 mg of the racemate from Example 13 by Method 2D gave 50 mg of the title compound from Example 14 (enantiomer 1) and 47 mg of the title compound from Example 15 (enantiomer 2).

HPLC (Method 2E): Rₜ=4.83 min, >99.5% ee; MS (ESIpos): m/z=457 [M+H]⁺.

### Example 15

### {3-[3-(2-Ethoxyethyl)-1,2,4-oxadiazol-5-yl]-5-(4-ethylphenyl)piperidin-1-yl}(4-hydroxypiperidin-1-yl)methanone [enantiomerically pure cis isomer]

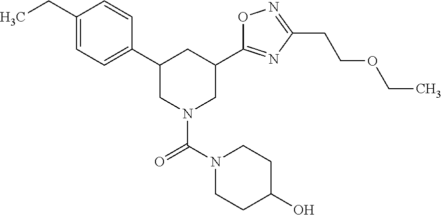

Enantiomer separation of 113 mg of the racemate from Example 13 by Method 2D gave 50 mg of the title compound from Example 14 (enantiomer 1) and 47 mg of the title compound from Example 15 (enantiomer 2).

HPLC (Method 2E): Rₜ=5.52 min, >99.5% ee; MS (ESIpos): m/z=457 [M+H]⁺.

### Example 16

### {3-[3-(2-Ethoxyethyl)-1,2,4-oxadiazol-5-yl]-5-[4-(trifluoromethyl)phenyl]piperidin-1-yl}(4-hydroxypiperidin-1-yl)methanone [racemic cis isomer]

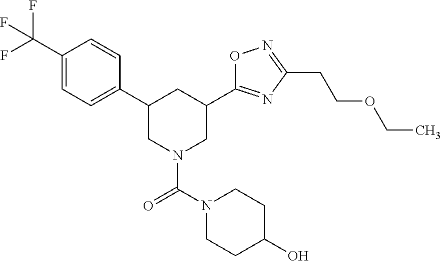

According to General Method 2, 100 mg (0.25 mmol) of the compound from Example 24A and 54 mg (0.38 mmol, 1.5 eq.) of the compound from Example 33A were converted. Yield: 82 mg (63% of theory).

HPLC (Method 7B): Rₜ=2.19 min; MS (ESIpos): m/z=497 [M+H]⁺;

¹H NMR (500 MHz, DMSO-d₆): δ=7.70 (d, 2H), 7.62-7.51 (m, 2H), 4.68 (d, 1H), 4.01-3.89 (m, 1H), 3.71 (t, 2H), 3.65-3.53 (m, 2H), 3.52-3.36 (m, 5H), 3.10-2.98 (m, 3H), 2.98-2.91 (m, 4H), 2.39-2.29 (m, 1H), 2.01 (q, 1H), 1.79-1.65 (m, 2H), 1.38-1.21 (m, 2H), 1.12-1.01 (m, 3H).

### Example 17

### {3-[3-(2-Ethoxyethyl)-1,2,4-oxadiazol-5-yl]-5-[4-(trifluoromethyl)phenyl]piperidin-1-yl}(4-hydroxypiperidin-1-yl)methanone [enantiomerically pure cis isomer]

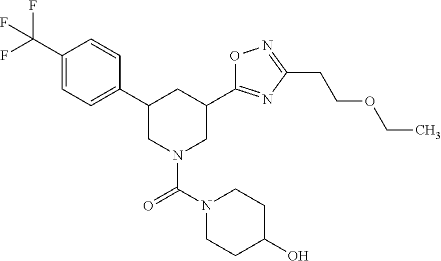

Enantiomer separation of 270 mg of the racemate from Example 16 by Method 2D gave 139 mg of the title compound from Example 17 (enantiomer 1) and 112 mg of the title compound from Example 18 (enantiomer 2).

HPLC (Method 2E): Rₜ=5.55 min, >99.5% ee; MS (ESIpos): m/z=497 [M+H]⁺.

### Example 18

### {3-[3-(2-Ethoxyethyl)-1,2,4-oxadiazol-5-yl]-5-[4-(trifluoromethyl)phenyl]piperidin-1-yl}(4-hydroxypiperidin-1-yl)methanone [enantiomerically pure cis isomer]

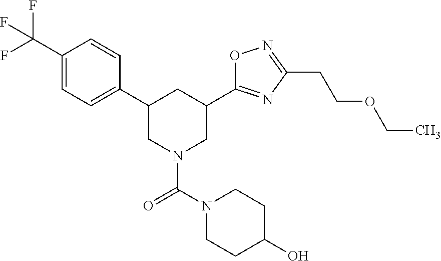

Enantiomer separation of 270 mg of the racemate from Example 16 by Method 2D gave 139 mg of the title compound from Example 17 (enantiomer 1) and 112 mg of the title compound from Example 18 (enantiomer 2).

HPLC (Method 2E): Rₜ=12.72 min, >99.5% ee; MS (ESIpos): m/z=497 [M+H]⁺.

### Example 19

### {3-[3-(2-Ethoxyethyl)-1,2,4-oxadiazol-5-yl]-5-[4-(trifluoromethoxy)phenyl]piperidin-1-yl}(4-hydroxypiperidin-1-yl)methanone [racemic cis isomer]

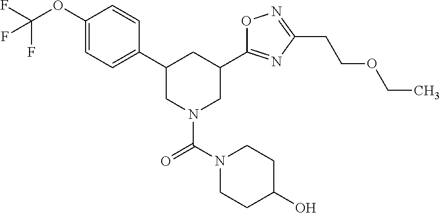

According to General Method 3, 350 mg (0.70 mmol) of the compound from Example 17A and 186 mg (1.41 mmol, 2.0 eq.) of the compound from Example 33A were converted. Yield: 81 mg (33% of theory).

LC-MS (Method 6B): Rₜ=1.07 min; MS (ESIpos): m/z=513 [M+H]⁺;

¹H NMR (500 MHz, DMSO-d₆): δ=7.46 (d, 2H), 7.33 (d, 2H), 4.67 (d, 1H), 4.00-3.90 (m, 1H), 3.71 (t, 2H), 3.64-3.52 (m, 2H), 3.51-3.36 (m, 5H), 3.07-2.85 (m, 5H), 2.32 (d, 1H), 2.04-1.90 (m, 1H), 1.78-1.64 (m, 2H), 1.38-1.23 (m, 2H), 1.07 (t, 3H).

### B) ASSESSMENT OF PHYSIOLOGICAL ACTIVITY

### Abbreviations

BSA bovine serum albumin
DMEM Dulbecco's Modified Eagle Medium
EGTA ethylene glycol-bis(2-aminoethyl)-N,N,N′,N′-tetraacetic acid
FCS fetal calf serum
HEPES 4-(2-hydroxyethyl)-1-piperazineethanesulphonic acid
[3H]haTRAP tritiated high affinity thrombin receptor activating peptide
PRP platelet-rich plasma

The suitability of the inventive compounds for treating thromboembolic disorders can be demonstrated in the following assay systems:

### 1.) In Vitro Assays

### 1.a) Cellular Functional In Vitro Test

A recombinant cell line is used to identify antagonists of the human protease activated receptor 1 (PAR-1) and to quantify the activity of the substances described herein. The cell is originally derived from a human embryonal kidney cell (HEK293; ATCC: American Type Culture Collection, Manassas, Va. 20108, USA). The test cell line constitutively expresses a modified form of the calcium-sensitive photoprotein aequorin which, after reconstitution with the cofactor coelenterazine, emits light when the free calcium concentration in the inner mitochondrial compartment is increased (Rizzuto R, Simpson A W, Brini M, Pozzan T.; Nature 1992, 358, 325-327). Additionally, the cell stably expresses the endogenous human PAR-1 receptor and the endogenous purinergic receptor P2Y2. The resulting PAR-1 test cell responds to stimulation of the endogenous PAR-1 or P2Y2 receptor with an intracellular release of calcium ions, which can be quantified through the resulting aequorin luminescence with a suitable luminometer (Milligan G, Marshall F, Rees S, Trends in Pharmacological Sciences 1996, 17, 235-237).

For the testing of the substance specificity, the effect thereof after activation of the endogenous PAR-1 receptor is compared with the effect after activation of the endogenous purinergic P2Y2 receptor which utilizes the same intracellular signal path.

Test procedure: The cells are plated out two days (48 h) before the test in culture medium (DMEM F12, supplemented with 10% FCS, 2 mM glutamine, 20 mM HEPES, 1.4 mM pyruvate, 0.1 mg/ml gentamycin, 0.15% Na bicarbonate; BioWhittaker Cat. #BE04-687Q; B-4800 Verviers, Belgium) in 384-well microtitre plates and kept in a cell incubator (96% atmospheric humidity, 5% v/v CO₂, 37° C.). On the day of the test, the culture medium is replaced by a tyrode solution (in mM: 140 sodium chloride, 5 postassium chloride, 1 magnesium chloride, 2 calcium chloride, 20 glucose, 20 HEPES), which additionally contains the cofactor coelenterazine (25 μM) and glutathione (4 mM), and the microtitre plate is then incubated for a further 3-4 hours. The test substances are then pipetted onto the microtitre plate, and 5 minutes after the transfer of the test substances into the wells of the microtitre plate the plate is transferred into the luminometer, a PAR-1 agonist concentration which corresponds to EC₅₀ is added and the resulting light signal is immediately measured in the luminometer. To distinguish an antagonist substance action from a toxic action, the endogenous purinergic receptor is immediately subsequently activated with agonist (ATP, final concentration 10 μM) and the resulting light signal is measured. The results are shown in Table A:

TABLE A

| Example No. | IC₅₀ [nM] |
|---|---|
| 7 | 34 |
| 9 | 28 |
| 15 | 8.6 |

### 1.b) PAR-1 Receptor Binding Assay

Platelet membranes are incubated with 12 nM [3H]haTRAP and test substance in different concentrations in a buffer (50 mM Tris pH 7.5, 10 mM magnesium chloride, 1 mM EGTA, 0.1% BSA) at room temperature for 80 min. Then the mixture is transferred to a filter plate and washed twice with buffer. After addition of scintillation liquid, the radioactivity on the filter is measured in a beta counter.

### 1.c) Platelet Aggregation in Plasma

To determine the platelet aggregation, blood from healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood). To obtain platelet-rich plasma, the citrated whole blood is centrifuged at 140 g for 20 min.

For the aggregation measurements, aliquots of the platelet-rich plasma with increasing concentrations of test substance are incubated at 37° C. for 10 min. Subsequently, aggregation is triggered by addition of a thrombin receptor agonist (TRAP6, SFLLRN) in an aggregometer and determined at 37° C. by means of the turbidimetry method according to Born (Born, G. V. R., Cross M. J., The Aggregation of Blood Platelets; J. Physiol. 1963, 168, 178-195). The SFLLRN concentration leading to maximum aggregation is, if appropriate, determined individually for each donor.

To calculate the inhibitory effect, the maximum increase of light transmission (amplitude of the aggregation curve in %) is determined within 5 minutes after addition of the agonist in the presence and absence of test substance, and the inhibition is calculated. The inhibition curves are used to calculate the concentration which inhibits aggregation by 50%.

### 1.d) Platelet Aggregation in Buffer

To determine platelet aggregation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood). To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1000 g for 10 minutes. The platelet pellet is resuspended with wash buffer and centrifuged at 1000 g for 10 minutes. The platelets are resuspended in incubation buffer and adjusted to 200 000 cells/μl. Prior to the start of the test, calcium chloride and magnesium chloride, final concentration in each case 2 mM (2M stock solution, dilution 1:1000), are added. Note: in the case of ADP-induced aggregation, only calcium chloride is added. The following agonists can be used: TRAP6-trifluoroacetate salt, collagen, human α-thrombin and U-46619. For each donor, the concentration of the agonist is tested.

Test procedure: 96-well microtitre plates are used. The test substance is diluted in DMSO, and 2 ml per well are initially charged. 178 μl of platelet suspension are added, and the mixture is preincubated at room temperature for 10 minutes. 20 μl of agonist are added, and the measurement in the Spectramax, OD 405 nm, is started immediately. Kinetics are determined in 11 measurements of 1 minute each. Between the measurements, the mixture is shaken for 55 seconds.

### 1.e) Platelet Aggregation in Fibrinogen-Depleted Plasma

To determine platelet aggregation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part of citrate+9 parts of blood).

Preparation of fibrinogen-depleted plasma: to obtain low-platelet plasma, the citrate whole blood is centrifuged at 140 g for 20 min. The low-platelet plasma is admixed in a ratio of 1:25 with reptilase (Roche Diagnostic, Germany) and inverted cautiously. This is followed by incubation at 37° C. in a water bath for 10 min, followed directly by incubation on ice for 10 min. The plasma/reptilase mixture is centrifuged at 1300 g for 15 min, and the supernatant (fibrinogen-depleted plasma) is obtained.

Platelet isolation: To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1300 g for 10 minutes. The platelet pellet is resuspended with wash buffer and centrifuged at 1300 g for 10 minutes. The platelets are resuspended in incubation buffer and adjusted to 400 000 cells/μl, and calcium chloride solution is added with a final concentration of 5 mM (dilution 1/200).

For the aggregation measurements, aliquots (98 μl of fibrinogen-depleted plasma and 80 μl of platelet suspension) are incubated with increasing concentrations of test substance at RT for 10 min. Subsequently, aggregation is triggered by addition of human alpha thrombin in an aggregometer and determined at 37° C. by means of the turbidimetry method according to Born (Born, G. V. R., Cross M. J., The Aggregation of Blood Platelets; J. Physiol. 1963, 168, 178-195). The alpha thrombin concentration which just leads to the maximum aggregation is determined individually for each donor.

To calculate the inhibitory effect, the increase in the maximum light transmission (amplitude of the aggregation curve in %) is determined within 5 minutes after addition of the agonist in the presence and absence of test substance, and the inhibition is calculated. The inhibition curves are used to calculate the concentration which inhibits aggregation by 50%.

### 1.f) Stimulation of Washed Platelets and Analysis in Flow Cytometry

Isolation of washed platelets: Human whole blood is obtained by venipuncture from voluntary donors and transferred to monovettes (Sarstedt, Nümbrecht, Germany) containing sodium citrate as anticoagulant (1 part sodium citrate 3.8%+9 parts whole blood). The monovettes are centrifuged at 90° rotations per minute and 4° C. for a period of 20 minutes (Heraeus Instruments, Germany; Megafuge 1.0RS). The platelet-rich plasma is carefully removed and transferred to a 50 ml Falcon tube. ACD buffer (44 mM sodium citrate, 20.9 mM citric acid, 74.1 mM glucose) is then added to the plasma. The volume of the ACD buffer corresponds to one quarter of the plasma volume. Centrifuging at 2500 rpm and 4° C. for ten minutes sediments the platelets. Thereafter, the supernatant is cautiously decanted off and discarded. The precipitated platelets are first cautiously resuspended in one millilitre of wash buffer (113 mM sodium chloride, 4 mM disodium hydrogenphosphate, 24 mM sodium dihydrogenphosphate, 4 mM potassium chloride, 0.2 mM ethylene glycol-bis(2-aminoethyl)-N,N,N′,N′-tetraacetic acid, 0.1% glucose) and then made up with wash buffer to a volume which corresponds to that of the amount of plasma. The wash procedure is repeated. The platelets are precipitated by another centrifugation at 2500 rpm and 4° C. for ten minutes and then carefully resuspended in one millilitre of incubation buffer (134 mM sodium chloride, 12 mM sodium hydrogencarbonate, 2.9 mM potassium chloride, 0.34 mM sodium dihydrogencarbonate, 5 mM HEPES, 5 mM glucose, 2 mM calcium chloride and 2 mM magnesium chloride) and adjusted with incubation buffer to a concentration of 300 000 platelets per μl.

Staining and stimulation of the human platelets with human α-thrombin in the presence or absence of a PAR-1 antagonist: The platelet suspension is preincubated with the substance to be tested or the appropriate solvent at 37° C. for 10 minutes (Eppendorf, Germany; Thermomixer Comfort). Platelet activation is triggered by addition of the agonist (0.5 μM or 1 μM α-thrombin; Kordia, the Netherlands, 3281 NIH units/mg; or 30 μg/ml of thrombin receptor activating peptide (TRAP6); Bachem, Switzerland) at 37° and with shaking at 500 rpm. One 50 μl aliquot of removed at each of 0, 1, 2.5, 5, 10 and 15 minutes, and transferred into one millilitre of singly concentrated CellFix™ solution (Becton Dickinson Immunocytometry Systems, USA). To fix the cells, they are incubated in the dark at 4° C. for 30 minutes. The platelets are precipitated by centrifuging at 600 g and 4° C. for ten minutes. The supernatant is discarded and the platelets are resuspended in 400 μl CellWash™ (Becton Dickinson Immunocytometry Systems, USA). One aliquot of 100 μl is transferred to a new FACS tube. 1 μl of the platelet-identifying antibody and 1 μl of the activation state-detecting antibody are made up to a volume of 100 μl with CellWash™. This antibody solution is then added to the platelet suspension and incubated in the dark at 4° C. for 20 minutes. After staining, the reaction volume is increased by addition of a further 400 μl of CellWash™.

A fluorescein isothiocyanate-conjugated antibody directed against human glycoprotein IIb (CD41) (Immunotech Coulter, France; Cat. No. 0649) is used to identify the platelets. With the aid of the phycoerythrin-conjugated antibody directed against human glycoprotein P-selectin (Immunotech Coulter, France; Cat. No. 1759), it is possible to determine the activation state of the platelets. P-Selectin (CD62P) is localized in the α-granules of resting platelets. However, following in vitro or in vivo stimulation, it is translocalized to the external plasma membrane.

Flow cytometry and data evaluation: The samples are analysed in the FACSCalibur™ Flow Cytometry System instrument from Becton Dickinson Immunocytometry Systems, USA, and evaluated and graphically presented with the aid of the CellQuest software, Version 3.3 (Becton Dickinson Immunocytometry Systems, USA). The extent of platelet activation is determined by the percentage of CD62P-positive platelets (CD41-positive events). From each sample, 10 000 CD41-positive events are counted.

The inhibitory effect of the substances to be tested is calculated via the reduction in platelet activation, which relates to the activation by the agonist.

### 1.g) Platelet Aggregation Measurement Using the Parallel-Plate Flow Chamber

To determine platelet activation, blood of healthy volunteers of both genders, who had not received any platelet aggregation-influencing medication for the last ten days, is used. The blood is taken up into monovettes (Sarstedt, Nümbrecht, Germany) which contain, as anticoagulant, sodium citrate 3.8% (1 part citrate+9 parts blood). To obtain platelet-rich plasma, the citrated whole blood is centrifuged at 140 g for 20 min. One quarter of the volume of ACD buffer (44.8 mM sodium citrate, 20.9 mM citric acid, 74.1 mM glucose and 4 mM potassium chloride) is added to the PRP, and the mixture is centrifuged at 1000 g for 10 minutes. The platelet pellet is resuspended in wash buffer and centrifuged at 1000 g for 10 minutes. For the perfusion study, a mixture of 40% erythrocytes and 60% washed platelets (200 000/μl) is prepared and suspended in HEPES-tyrode buffer. Platelet aggregation under flow conditions is measured using the parallel-plate flow chamber (B. Nieswandt et al., EMBO J. 2001, 20, 2120-2130; C. Weeterings, Arterioscler Thromb. Vasc. Biol. 2006, 26, 670-675; J J Sixma, Thromb. Res. 1998, 92, 43-46). Glass slides are wetted with 100 μl of a solution of human α-thrombin (dissolved in Tris buffer) at 4° C. overnight (α-thrombin in different concentrations, e.g. 10 to 50 mg/ml) and then blocked using 2% BSA.

Reconstituted blood is passed over the thrombin-wetted glass slides at a constant flow rate (for example a shear rate of 300/second) for 5 minutes and observed and recorded using a microscope video system. The inhibitory activity of the substances to be tested is determined morphometrically via the reduction of platelet aggregate formation. Alternatively, the inhibition of the platelet activation can be determined by flow cytometry, for example via p-selectin expression (CD62p) (see Method 1.f).

### 2.) Ex Vivo Assay

### 2.a) Platelet Aggregation (Primates, Guinea Pigs)

Awake or anaesthetized guinea pigs or primates are treated orally, intravenously or intraperitoneally with test substances in suitable formulations. As a control, other guinea pigs or primates are treated in an identical manner with the corresponding vehicle. Depending on the mode of administration, blood of the deeply anaesthetized animals is obtained by puncture of the heart or of the aorta for different periods of time. The blood is taken up into monovettes (Sarstedt, Nümbrecht, Germany) which, as anticoagulant, contain sodium citrate 3.8% (1 part citrate solution+9 parts blood). To obtain platelet-rich plasma, the citrated whole blood is centrifuged at 140 g for 20 min.

Aggregation is triggered by addition of a thrombin receptor agonist (TRAP6, SFLLRN, 50 μg/ml; in each experiment, the concentration is determined for each animal species) in an aggregometer and determined by means of the turbidimetry method according to Born (Born, G. V. R., Cross M. J., The Aggregation of Blood Platelets; J. Physiol. 1963, 168, 178-195) at 37° C.

To measure the aggregation, the maximum increase in the light transmission (amplitude of the aggregation curve in %) is determined within 5 minutes after addition of the agonist. The inhibitory effect of the administered test substances in the treated animals is calculated via the reduction in aggregation, based on the mean of the control animals.

### 2.b) Platelet Aggregation and Activation Measurement in the Parallel-Plate Flow Chamber (Primates)

Awake or anaesthetized primates are treated orally, intravenously or intraperitoneally with test substances in suitable formulations. As a control, other animals are treated in an identical manner with the corresponding vehicle. According to the mode of administration, blood is obtained from the animals by venipuncture for different periods of time. The blood is transferred into monovettes (Sarstedt, Nümbrecht, Germany) which, as anticoagulant, contain sodium citrate 3.8% (1 part citrate solution+9 parts blood). Alternatively, non-anticoagulated blood can be taken with neutral monovettes (Sarstedt). In both bases, the blood is admixed with Pefabloc FG (Pentapharm, final concentration 3 mM) to prevent fibrin clot formation.

Citrated whole blood is recalcified before the measurement by adding CaCl₂ solution (final Ca⁺⁺ concentration 5 mM). Non-anticoagulated blood is introduced directly into the parallel-plate flow chamber for measurement. The measurement of platelet activation is conducted by morphometry or flow cytometry in the collagen-coated parallel-plate flow chamber, as described in Method 1.h).

### 3.) In Vivo Assays

### 3.a) Thrombosis Models

The inventive compounds can be studied in thrombosis models in suitable animal species in which thrombin-induced platelet aggregation is mediated via the PAR-1 receptor. Suitable animal species are guinea pigs and, in particular, primates (cf.: Lindahl, A. K., Scarborough, R. M., Naughton, M. A., Harker, L. A., Hanson, S. R., Thromb Haemost 1993, 69, 1196; Cook J J, Sitko G R, Bednar B, Condra C, Mellott M J, Feng D-M, Nutt R F, Shager J A, Gould R J, Connolly T M, Circulation 1995, 91, 2961-2971; Kogushi M, Kobayashi H, Matsuoka T, Suzuki S, Kawahara T, Kajiwara A, Hishinuma I, Circulation 2003, 108 Suppl. 17, IV-280; Derian C K, Damiano B P, Addo M F, Darrow A L, D'Andrea M R, Nedelman M, Zhang H-C, Maryanoff B E, Andrade-Gordon P, J. Pharmacol. Exp. Ther. 2003, 304, 855-861). Alternatively, it is possible to use guinea pigs which have been pretreated with inhibitors of PAR-3 and/or PAR-4 (Leger A J et al., Circulation 2006, 113, 1244-1254), or transgenic PAR-3- and/or PAR-4-knockdown guinea pigs.

### 3.b) Impaired Coagulation and Organ Dysfunction in the Case of Disseminated Intravasal Coagulation (DIC)

The inventive compounds can be tested in models of DIC and/or sepsis in suitable animal species. Suitable animal species are guinea pigs and, in particular, primates, and for the study of endothelium-mediated effects also mice and rats (cf.: Kogushi M, Kobayashi H, Matsuoka T, Suzuki S, Kawahara T, Kajiwara A, Hishinuma I, Circulation 2003, 108 Suppl. 17, IV-280; Derian C K, Damiano B P, Addo M F, Darrow A L, D'Andrea M R, Nedelman M, Zhang H—C, Maryanoff B E, Andrade-Gordon P, J. Pharmacol. Exp. Ther. 2003, 304, 855-861; Kaneider N C et al., Nat Immunol, 2007, 8, 1303-12; Camerer E et al., Blood, 2006, 107, 3912-21; Riewald M et al., J Biol Chem, 2005, 280, 19808-14.). Alternatively, it is possible to use guinea pigs which have been pretreated with inhibitors of PAR-3 and/or PAR-4 (Leger A J et al., Circulation 2006, 113, 1244-1254), or transgenic PAR-3- and/or PAR-4-knockdown guinea pigs.

### 3.b.1) Thrombin-Antithrombin Complexes

Thrombin-antithrombin complexes (referred to hereinafter as “TAT”) are a measure of the thrombin formed endogenously by coagulation activation. TATs are determined via an ELISA assay (Enzygnost TAT micro, Dade-Behring). Plasma is obtained from citrated blood by centrifugation. 50 μl of TAT sample buffer are added to 50 μl of plasma, shaken briefly and incubated at room temperature for 15 min. The samples are filtered with suction, and the well is washed 3 times with wash buffer (300 μl/well). Between the wash steps, the plate is tapped to remove any residual wash buffer. Conjugate solution (100 μl) is added and the mixture is incubated at room temperature for 15 min. The samples are filtered with suction, and the well is washed 3 times with wash buffer (3000 μl/well). Chromogenic substrate (1000 μl/well) is then added, the mixture is incubated in the dark at room temperature for 30 min, stop solution (100 μl/well) is added, and the development of colour at 492 nm is measured (Safire plate reader).

### 3.b.2) Parameters of Organ Dysfunction

Various parameters are determined, which allow conclusions to be drawn with respect to the restriction of function of various internal organs owing to the administration of LPS, and the therapeutic effect of test substances to be estimated. Citrated blood or, if appropriate, lithium heparin blood, is centrifuged, and the plasma is used to determine the parameters. Typically, the following parameters are determined: creatinine, urea, aspartate aminotransferase (AST), alanine aminotransferase (ALT), total bilirubin, lactate dehydrogenase (LDH), total protein, total albumin and fibrinogen. The values give information regarding kidney function, liver function, cardiovascular function and vascular function.

### 3.b.3) Parameters of Inflammation

The extent of the inflammatory reaction triggered by endotoxin can be demonstrated by the rise in inflammation mediators, for example interleukins (1, 6, 8 and 10), tumour necrosis factor alpha or monocyte chemoattractant protein-1, in the plasma. ELISAs or the Luminex system can be used for this purpose.

### 3.c) Antitumour Activity

The inventive compounds can be tested in models of cancer, for example in the human breast cancer model in immunodeficient mice (cf.: S. Even-Ram et. al., Nature Medicine, 1988, 4, 909-914).

### 3.d) Antiangiogenetic Activity

The inventive compounds can be tested in in vitro and in vivo models of angiogenesis (cf.: Caunt et al., Journal of Thrombosis and Haemostasis, 2003, 10, 2097-2102; Haralabopoulos et al., Am J Physiol, 1997, C239-C245; Tsopanoglou et al., JBC, 1999, 274, 23969-23976; Zania et al., JPET, 2006, 318, 246-254).

### 3.e) Blood Pressure- and Pulse-Modulating Activity

The inventive compounds can be tested in in vivo models for their effect on arterial blood pressure and heart rate. To this end, rats (for example Wistar) are provided with implantable radiotelemetry units, and an electronic data acquisition and storage system (Data Sciences, MN, USA) consisting of a chronically implantable transducer/transmitter unit in combination with a liquid-filled catheter is employed. The transmitter is implanted into the peritoneal cavity, and the sensor catheter is positioned in the descending aorta. The inventive compounds can be administered (for example orally or intravenously). Prior to the treatment, the mean arterial blood pressure and the heart rate of the untreated and treated animals are measured, and it is ensured that they are in the range of about 131-142 mmHg and 279-321 beats/minute. PAR-1-activating peptide (SFLLRN; for example doses between 0.1 and 5 mg/kg) is administered intravenously. Blood pressure and heart rate are measured at various time intervals and durations with and without PAR-1-activating peptide and with and without one of the inventive compounds (cf.: Cicala C et al., The FASEB Journal, 2001, 15, 1433-5; Stasch J P et al., British Journal of Pharmacology 2002, 135, 344-355).

### 4.) Determination of the Solubility

### Preparation of the Starting Solution (Original Solution):

At least 1.5 mg of the test substance are weighed out accurately into a wide-mouth 10 mm screw V-vial (from Glastechnik Gräfenroda GmbH, Art. No. 8004-WM-H/V15μ) with fitting screw cap and septum, DMSO is added to a concentration of 50 mg/ml and the vial is vortexed for 30 minutes.

### Preparation of the Calibration Solutions:

The pipetting steps necessary are effected in 1.2 ml 96-well deep well plates (DWP) with the aid of a liquid-handling robot. The solvent used is a mixture of acetonitrile/water 8:2.

Preparation of the starting solution of calibration solutions (stock solution): 833 μl of the solvent mixture are added to 10 μl of the original solution (concentration=600 μg/ml), and the mixture is homogenized. 1:100 dilutions in separate DWPs are prepared from each test substance, and these are homogenized in turn.

Calibration solution 5 (600 ng/ml): 270 μl of the solvent mixture are added to 30 μl of the stock solution, and the mixture is homogenized.

Calibration solution 4 (60 ng/ml): 270 μl of the solvent mixture are added to 30 μl of the calibration solution 5, and the mixture is homogenized.

Calibration solution 3 (12 ng/ml): 400 μl of the solvent mixture are added to 100 μl of the calibration solution 4, and the mixture is homogenized.

Calibration solution 2 (1.2 ng/ml): 270 μl of the solvent mixture are added to 30 μl of the calibration solution 3, and the mixture is homogenized.

Calibration solution 1 (0.6 ng/ml): 150 μl of the solvent mixture are added to 150 μl of the calibration solution 2, and the mixture is homogenized.

### Preparation of the Sample Solutions:

The pipetting steps necessary are effected in 1.2 ml 96-well DWPs with the aid of a liquid-handling robot. 1000 μl of PBS buffer pH 6.5 are added to 10.1 μl of the stock solution. (PBS buffer pH 6.5: 61.86 g sodium chloride, 39.54 g sodium dihydrogen phosphate and 83.35 g 1 N sodium hydroxide solution are weighed into a 1 litre standard flask and made up to the mark with water, and the mixture is stirred for about 1 hour. 500 ml of this solution are introduced into a 5 litre standard flask and made up to the mark with water. The pH is adjusted to 6.5 using 1 N sodium hydroxide solution.)

### Procedure:

The pipetting steps necessary are effected in 1.2 ml 96-well DWPs with the aid of a liquid-handling robot. The sample solutions prepared in this manner are shaken at 1400 rpm and at 20° C. using a variable temperature shaker for 24 hours. 180 μl are taken from each of these solutions and transferred into Beckman Polyallomer centrifuge tubes. These solutions are centrifuged at about 223 000×g for 1 hour. From each sample solution, 100 μl of the supernatant are removed and diluted 1:10 and 1:1000 with PBS buffer 6.5.

### Analysis:

The samples are analysed by means of HPLC/MS-MS. The test compound is quantified by means of a five-point calibration curve. The solubility is expressed in mg/l. Analysis sequence: 1) blank (solvent mixture); 2) calibration solution 0.6 ng/ml; 3) calibration solution 1.2 ng/ml; 4) calibration solution 12 ng/ml; 5) calibration solution 60 ng/ml; 6) calibration solution 600 ng/ml; 7) blank (solvent mixture); 8) sample solution 1:1000; 9) sample solution 1:10.

### HPLC/MS-MS Method:

HPLC: Agilent 1100, quat. pump (G1311A), autosampler CTC HTS PAL, degasser (G1322A) and column thermostat (G1316A); column: Oasis HLB 20 mm×2.1 mm, 25μ; temperature: 40° C.; eluent A: water+0.5 ml of formic acid/1; eluent B: acetonitrile+0.5 ml of formic acid/1; flow rate: 2.5 ml/min; stop time 1.5 min; gradient: 0 min 95% A, 5% B; ramp: 0-0.5 min 5% A, 95% B; 0.5-0.84 min 5% A, 95% B; ramp: 0.84-0.85 min 95% A, 5% B; 0.85-1.5 min 95% A, 5% B.

MS/MS: WATERS Quattro Micro Tandem MS/MS; Z-Spray API interface; HPLC-MS inlet splitter 1:20; measurement in the ESI mode.

### C) WORKING EXAMPLES OF PHARMACEUTICAL COMPOSITIONS

The inventive substances can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% solution (m/m) of the PVP in water. The granules are dried and then mixed with the magnesium stearate for 5 min. This mixture is compressed in a conventional tablet press (see above for tablet format).

### Oral Suspension:

### Composition:

1000 mg of the compound of Example 1, 1000 mg of ethanol (96%), 400 mg of Rhodigel (xanthan gum) (from FMC, USA) and 99 g of water.

A single dose of 100 mg of the inventive compound corresponds to 10 ml of oral suspension.

### Production:

The Rhodigel is suspended in ethanol, and the compound of Example 1 is added to the suspension. The water is added while stirring. The mixture is stirred for approx. 6 h until the Rhodigel has finished swelling.

### Intravenously Administrable Solution:

### Composition:

1 mg of the compound of Example 1, 15 g of polyethylene glycol 400 and 250 g of water for injections.

### Production:

The compound of Example 1 is dissolved together with polyethylene glycol 400 by stirring in the water. The solution is sterile-filtered (pore diameter 0.22 μm) and dispensed under aseptic conditions into heat-sterilized infusion bottles. The latter are closed with infusion stoppers and crimped caps.

1. A compound of the formula 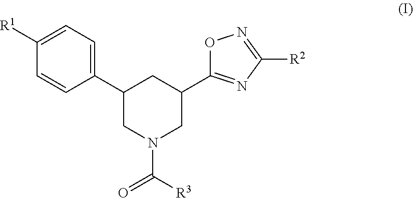
in which
R¹ is trifluoromethyl, trifluoromethoxy or ethyl,
R² is 2-methoxyeth-1-yl, 2-ethoxyeth-1-yl or cyclopropyl,
R³ is a group of the formula 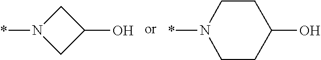
where
* is the point of attachment to the carbonyl group,
or one of its salts, its solvates or the solvates of its salts.

2. A compound according to claim 1, wherein
R¹ is trifluoromethyl or ethyl,
R² is 2-methoxyeth-1-yl or cyclopropyl,
R³ is a group of the formula 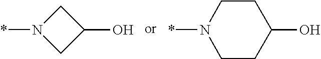
where
* is the point of attachment to the carbonyl group,
or one of its salts, its solvates or the solvates of its salts.

3. A compound according to claim 1, wherein
R¹ is trifluoromethoxy or ethyl,
R² is 2-ethoxyeth-1-yl,
R³ is a group of the formula 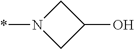
where
* is the point of attachment to the carbonyl group,
or one of its salts, its solvates or the solvates of its salts.

4. A compound according to claim 1, wherein the phenyl substituent and the 1,2,4-oxadiazol-5-yl substituent bonded to the piperidine ring, are in cis positions to one another.

5. A process for preparing a compound of the formula (I) or one of its salts, its solvates or the solvates of its salts according to claim 1, wherein either
[A] a compound of the formula 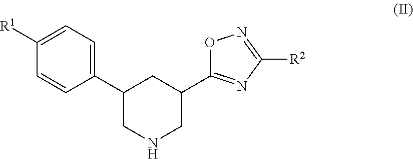
in which
R¹ and R² are each as defined in claim 1
is reacted with a compound of the formula 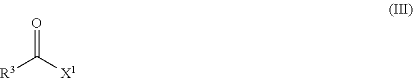
in which
R³ is as defined in claim 1 and
X¹ is halogen, preferably bromine or chlorine, or hydroxyl,
or
[B] a compound of the formula (II) is reacted in the first stage with 4-nitrophenyl chloroformate and in the second stage with a compound of the formula
R³—H (IV)
in which
R³ is as defined in claim 1,
or
[C] a compound of the formula 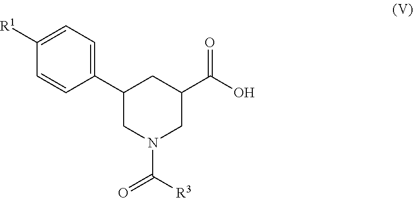
in which
R¹ and R³ are each as defined in claim 1,
is reacted with a compound of the formula 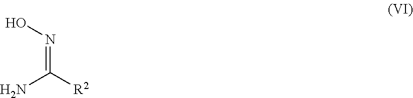
in which
R² is as defined in claim 1.

6. (canceled)

7. (canceled)

8. (canceled)

9. (canceled)

10. A pharmaceutical composition comprising a compound according to claim 1 in combination with an inert, non-toxic, pharmaceutically acceptable excipient.

11. A pharmaceutical composition comprising a compound according to claim 1 in combination with a further active ingredient.

12. (canceled)

13. A method for the treatment and/or prophylaxis of a thromboembolic disorder comprising administering to a human or animal in need thereof an anticoagulatory amount of a compound according to claim 1.

14. A method for the prevention of blood coagulation in vitro, comprising adding to blood ex vivo an anticoagulatory amount of a compound according to claim 1.

15. A method for the treatment and/or prophylaxis of a cardiovascular disorder comprising administering to a human or animal in need thereof an effective amount of a compound according to claim 1.

16. A method for the treatment and/or prophylaxis of a tumour disorder comprising administering to a human or animal in need thereof an effective amount of a compound according to claim 1.

17. A method for the treatment and/or prophylaxis of a thromboembolic disorder comprising administering to a human or animal in need thereof an anticoagulatory amount of a pharmaceutical composition according to claim 10.

18. A method for the treatment and/or prophylaxis of a cardiovascular disorder comprising administering to a human or animal in need thereof an anticoagulatory amount of a pharmaceutical composition according to claim 10.

19. A method for the treatment and/or prophylaxis of a tumour disorder comprising administering to a human or animal in need thereof an anticoagulatory amount of a pharmaceutical composition according to claim 10.
